(19) United States
(12) Patent Application Publication
(10) Pub. No.: US 2024/0245346 A1
(43) Pub. Date: Jul. 25, 2024

(54) IMAGE ANALYSIS USING SKIN WOUND FACTORS

(71) Applicant: Precision Healing, Inc., Newton, MA (US)

(72) Inventors: David B Strasfeld, Somerville, MA (US); Ryan Daniel Williams, Somerville, MA (US); Ira M Herman, Boston, MA (US); W David Lee, Brookline, MA (US)

(73) Assignee: Precision Healing, Inc., Newton, MA (US)

(21) Appl. No.: 18/599,306

(22) Filed: Mar. 8, 2024

Related U.S. Application Data
(63) Continuation-in-part of application No. 17/564,318, filed on Dec. 29, 2021.
Application No. 17/564,318 is a continuation-in-part of application No. 17/155,141, filed on Jan. 22, 2021.
(60) Provisional application No. 63/550,096, filed on Feb. 6, 2024.
(60) Provisional application No. 63/451,247, filed on Mar. 10, 2023.
(60) Provisional application No. 63/132,541, filed on Dec. 31, 2020.
(60) Provisional application No. 62/964,969, filed on Jan. 23, 2020.

(51) Int. Cl. A61B 5/00; A61B 5/01

(52) U.S. Cl. CPC A61B 5/445; A61B 5/0071; A61B 5/015; A61B 5/443

(57) ABSTRACT

Disclosed techniques include image analysis using skin wound factors. One or more skin wound images are obtained for an individual. The skin wound images provide light spectrum information that includes fluorescence and reflectance characteristics. The light spectrum information includes infrared thermal characteristics. Processors are used to analyze the skin wound images to determine metrics for skin wound factors, based on the light spectrum information. The skin wound factors comprise infection, inflammation, granulation, topology, and epithelialization. An epithelialization metric is included in determining skin wound topology. The skin wound topology comprises wound area, wound volume, wound depth, wound shape, wound edge definition, or wound topography. An output indicative of biophysical skin wound status is generated. The output is based on the analyzing of the skin wound factors. The output is augmented, based on analysis of the infrared thermal characteristics. A skin wound care plan is developed, based on the output.

### RELATED APPLICATIONS

This application claims the benefit of U.S. provisional patent applications “Image Analysis Using Skin Wound Factors” Ser. No. 63/451,247, filed Mar. 10, 2023, and “Wound Care Image Analysis Using A Smartphone” Ser. No. 63/550,096, filed Feb. 6, 2024.

This application is also a continuation-in-part of U.S. patent application “Multispectral Sample Analysis Using Absorption Signatures” Ser. No. 17/564,318, filed Dec. 29, 2021, which claims the benefit of U.S. provisional patent application “Multispectral Sample Analysis Using Fluorescence Signatures” Ser. No. 63/132,541, filed Dec. 31, 2020.

The U.S. patent application “Multispectral Sample Analysis Using Absorption Signatures” Ser. No. 17/564,318, filed Dec. 29, 2021 is also a continuation-in-part of U.S. patent application “Skin Diagnostics Using Optical Signatures” Ser. No. 17/155,141, filed Jan. 22, 2021, which claims the benefit of U.S. provisional patent applications “Systems and Methods for Wound Care Diagnostics and Treatment” Ser. No. 62/964,969, filed Jan. 23, 2020, and “Multispectral Sample Analysis Using Fluorescence Signatures” Ser. No. 63/132,541, filed Dec. 31, 2020.

Each of the foregoing applications is hereby incorporated by reference in its entirety.

### FIELD OF ART

This application relates generally to image analysis and more particularly to image analysis using skin wound factors.

### BACKGROUND

Natural, processed, and manufactured materials are essential to people everywhere. Materials are used for clothing, shelter, and vehicles, and for many other common and specialty items. Clothing materials are chosen to cover or protect, and for comfort, among other criteria. Clothing is worn to impress and to make strong statements about class, culture, origin, and beliefs. Structures, whether temporary, permanent, or mobile, are constructed based on purpose, design, and materials available. Vehicles ranging from pushcarts to rockets are manufactured to enable transportation. Objects are formed from materials that include a substance or a mixture of substances. Popular and widely used materials include wood, plastic, metal, fabric, and glass. The materials can be used in their natural forms or can be combined with other materials to form compounds, composites, alloys, or blends. The constituents of a single material or a combination of materials can be identified by studying material properties. The properties include hardness, visual appearance, and weight; physical properties such as material state; and other physical properties such as the density and magnetic characteristics. The material properties also include chemical properties such as resistance and combustibility. The material properties further include mechanical properties such as malleability, ductility, and strength; and electrical properties such as conductivity and resistivity. The material properties can further include optical properties such as light transmissivity and absorptivity. Since each material has its own unique set of properties, the physical, chemical, mechanical, electrical, optical, and other responses of a material can be analyzed to characterize and identify the materials.

Industries including manufacturing, aerospace, and taxonomy, to name but a few, perform analysis and characterization tasks on various materials. These tasks are also applied to research and development applications. The tasks are used to identify one or more materials within a sample, to characterize new alloys or material compounds, and so on. The analysis and characterization tasks are also used to detect and identify the presence of unexpected materials within a material sample. The unexpected materials within a sample can include contaminants or impurities within materials. The presence of contaminants within materials is generally considered undesirable because the contaminants can contribute to substandard materials that cause systems made from the materials to fail. Another class of materials analysis, based on complex testing procedures and advanced testing techniques, is used to obtain detailed information about a material or sample. The detailed information can include identification of the chemical composition of the material. This latter class of materials analysis can require sophisticated laboratory equipment and advanced personnel training. Material surface topology and composition can be determined using a scanning electron microscope (SEM), which uses a beam of electrons to generate an image. A transmission electron microscope (TEM) can be used in crystalline defect analysis to predict material behavior and to find failure mechanisms. X-ray Diffraction (XRD) is also used to identify and characterize crystalline materials. These complicated and expensive tests, techniques, and pieces of equipment, which are usually available only in laboratories, can be used alone or in combination to characterize and identify unknown materials.

### SUMMARY

Techniques for wound care image analysis using skin wound factors are disclosed. The skin wound factors can be based on fluorescence characteristics and reflectance characteristics of skin wound images obtained for an individual. The skin wound images are based on a skin wound, an exudate sample associated with the skin wound, and so on. The sample is excited, illuminated, or irradiated by various wavelengths of light or light spectra. The light spectra can include light along the Red-Green-Blue (RGB) light wavelength spectrum, the infrared (IR) light spectrum, etc. This technique uses a range of light wavelengths across the electromagnetic spectrum to determine fluorescence characteristics and reflectance characteristics associated with the skin wound images. The fluorescence characteristics and the reflectance characteristics are determined in response to at least one excitation light wavelength. The reflectance characteristics can be determined in response to at least three additional light wavelengths. The light wavelengths can include infrared light, visible light, etc. Molecules of a skin wound, and the skin wound sample, fluoresce or reflect the light sources. The presence of fluorescence light wavelengths and reflected light wavelengths from the material sample can be detected by one or more RGB sensors and other sensors. The data collected by the RGB, IR, etc. sensors can be used to generate the skin wound images. The RGB sensors can comprise components of a broad-spectrum image sensor. The broad-spectrum sensor can employ an integrated, very low-cost Bayer filter, which enables the broad-spectrum image sensor to provide sensitivities to particular wavelengths, including light from frequencies which are visible to the human eye and those frequencies which are not. Various elements within a skin wound fluoresce or reflect different wavelengths of light when compared to one another. The image analysis can be used to differentiate skin wound analytes based on their spectral fluorescence and reflection signatures and characteristics. The image analysis is applied to the skin wound images to enable skin wound care. As disclosed, image analysis using skin wound factors, such as topology, inflammation, epithelialization, granulation, and infection, can reduce the complexity, cost, and deployment challenges of using specialized multispectral cameras, elaborate optical filters, and expensive filter wheels. The filter wheels, in particular, have orientation and alignment sensitivities which are difficult to manage. Further, the cameras, optical filters, and filter wheels require fixed, lab-only equipment placement. The providing of excitation light spectra, and the measuring output values of one or more RGB sensors, can be performed by a handheld, mobile device.

A method for image analysis is disclosed comprising: obtaining one or more skin wound images for an individual, wherein the skin wound images provide light spectrum information that includes fluorescence characteristics and reflectance characteristics; analyzing, using one or more processors, the skin wound images to determine metrics for skin wound factors, based on the light spectrum information, wherein the skin wound factors comprise infection, inflammation, granulation, topology, and epithelialization; and generating an output indicative of biophysical skin wound status, wherein the output is based on the analyzing of the skin wound factors. A five-factor technique comprises the various skin wound factors, and the five factors are used to generate an output indicative of biophysical skin wound status. The five factors include identifying wound topology, inflammation, epithelialization, granulation, and infection associated with the skin wound. The five factors can be analyzed within skin wound images to determine metrics for skin wound factors. In embodiments, the analysis includes identifying granulation within the material sample. The granulation is identified based on a measure of hemoglobin and collagen. In embodiments, the analysis includes identifying infection within the material sample. The infection is identified based on a measure of a porphyrin, pyoverdine, slough, eschar, or on an inflammation signature. In embodiments, identifying wound topology, inflammation, epithelialization, granulation, and infection comprises a five-factor biophysical material sample status.

In embodiments, the light spectrum information includes infrared thermal characteristics. The generated output that is indicative of biophysical skin wound status is augmented based on analysis of the infrared thermal characteristics. In embodiments, the analysis includes identifying a wound topology within the material sample. The wound topology includes the area, volume, depth, profile, texture, roughness, and so on of the wound. The wound topology is further identified based on a measure of granulation, slough, eschar, flavin adenine dinucleotide (FAD), and/or nicotinamide adenine dinucleotide plus hydrogen (NADH) in the wound sample. In embodiments, the analysis includes identifying inflammation within the skin wound sample image. The inflammation is identified based on a measure of heat, redness, and swelling in the material sample. In embodiments, the analysis includes identifying epithelialization within the material sample. The epithelialization is identified based on a measure of nicotinamide adenine dinucleotide (NAD), NADH, and/or FAD.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1A is a flow diagram for image analysis using skin wound factors.

FIG. 1B is a flow diagram for bandage sampling.

FIG. 2 is a high-level block diagram for five-factor analysis.

FIG. 3 shows a system block diagram for multispectral sample analysis.

FIG. 4 illustrates a system block diagram for using fluorescence signatures.

FIG. 5 is a graph showing fluorescence measurements.

FIG. 6 is a graph illustrating biochrome absorption.

FIG. 7 is a table showing biochromes and fluorescent channels.

FIG. 8 illustrates granulation identification.

FIG. 9 shows water identification.

FIG. 10 illustrates blood vessel identification.

FIG. 11 shows tissue oximetry.

FIG. 12 is a system block diagram for a lateral flow assay.

FIG. 13 is a system diagram for image analysis using skin wound factors.

### DETAILED DESCRIPTION

Techniques for wound care image analysis using skin wound factors are disclosed. The skin wound factors are based on both fluorescence characteristics and reflectance characteristics. The fluorescence characteristics and reflectance characteristics are included within light spectrum information provided by one or more skin wound images for an individual. The skin wound images are obtained for wound samples, exudate samples, and so on that are excited, illuminated, irradiated, etc. with a plurality of light wavelength sources. The light wavelength sources are based on one or more light spectra. The light spectra can be based on light along the Red-Green-Blue (RGB) light wavelength spectrum, an infrared (IR) spectrum, a near-infrared (NIR) spectrum, a far-infrared (FIR) spectrum, etc. This technique uses light from a range of wavelengths across the electromagnetic spectrum to determine light fluorescence and light reflectance by a skin wound sample. The fluorescence characteristics and reflectance characteristics of the skin wound are determined in response to one or more light spectrum sources. The light spectrum sources cause molecules of a skin wound sample to fluoresce and reflect light wavelengths. The light fluoresced and reflected by the skin wound sample can be detected by one or more RGB sensors. The RGB sensors can be components of a broad-spectrum image sensor. The broad-spectrum sensor can employ an integrated, very low-cost Bayer filter, which enables the broad-spectrum image sensor to provide sensitivities to particular wavelengths, including light from frequencies which are visible to the human eye and those which are not. Various materials, such as materials within a skin wound sample, fluoresce and reflect different wavelengths of light when compared to one another. However, imaging such as multispectral imaging can be used to differentiate materials based on their spectral fluorescence and reflectance signatures and characteristics. As disclosed, multispectral imaging based on fluorescence and reflectance can reduce the complexity, cost, and deployment challenges of using specialized multispectral cameras, elaborate optical filters, and expensive filter wheels. The filter wheels can have significant orientation and alignment sensitivities rendering the filter wheels difficult to implement. Further, the multispectral analysis can be performed without employing fixed, lab-only equipment placement. The providing of excitation and illumination light wavelengths and the measuring output values of one or more RGB sensors can be performed by a handheld, mobile device. The handheld, mobile device can be adapted for use on an outdoor-suitable vehicle, such as a drone aircraft, an autonomous vehicle, and so on.

Common filters can be used to spectrally resolve a fluorescence signal and a reflectance signal. Light spectrum information that includes the fluorescence signal and the reflectance signal can be spectrally resolved using filters associated with many widely available color digital imagers. An example of such filters includes the Red, Green, and Blue (RGB) Bayer filters integrated in typical, inexpensive RGB sensors that form the basis of common color digital imagers. These RGB sensors generally demonstrate peak blue sensitivity at 400-475 nm, peak green sensitivity at 475-580 nm, and peak red sensitivity at 580-750 nm. Excitation and illumination light sources at wavelengths near the edge of, inside of, or outside of the RGB visible light wavelength spectrum, which can be referred to as the extended RGB spectrum, can include wavelengths from 350 nm to 950 nm, for example. However, note that the definition of the exact wavelengths of visible light is somewhat subjective. For purposes of discussion, a visible light wavelength spectrum of about 425 nm to 725 nm is understood herein, although discrete wavelengths or wavelength ranges are used when possible. The excitation and illumination source wavelengths can, when used to illuminate a material sample, elicit fluorescence and reflectance responses from the material sample. The fluorescence and reflectance responses can be detected by one or more RGB sensors, and images generated based on the RGB sensor data. That is, a given material sample will fluoresce or reflect, with varying magnitudes, an impinging excitation, or illumination, source wavelength. The excitation and illumination sources may be chosen to have a relatively narrow, non-overlapping spectrum to avoid “crosstalk” excitation and illumination.

RGB sensors can be used in additional configurations to provide further information associated with a material sample such as skin wound samples discussed herein. In embodiments, two RGB sensors can be employed to provide stereoscopic imaging of a material sample. The stereoscopic imaging is valuable for feature detection, identification, and matching in a skin wound sample. The two RGB sensors can have polarization filters inserted over or in front of them to provide a measure of polarization in the images. For example, one sensor can be polarized in a vertical orientation, while the other sensor can be polarized in a horizontal orientation, thus providing 90° “cross-polarization” for the stereoscopic imaging. This cross-polarization allows for the isolation of specularly fluoresced and reflected, polarized photons based on comparison of the images taken from the two sensors. The cross-polarization can provide information critical to determining a state of a skin wound, healing progress, and so on.

The method of wound care image analysis using skin wound factors disclosed herein uses one or more ordinary, readily available Red-Green-Blue (RGB) sensors. The RGB sensor can detect multispectral fluorescence and reflectance responses of a skin wound to various wavelengths of light. The multispectral fluorescence and reflectance responses are captured in images for an individual. The images are analyzed to determine metrics for skin wound factors such as infection, inflammation, granulation, topology, and epithelialization. The RGB sensor is typically mass produced and has applications in low-cost technology that endeavors to detect light waves in the visible spectrum in a standard three-color, RGB palette suitable for digital processing. The RGB sensor typically employs an integrated Bayer filter applied during the manufacturing process of a CMOS, CCD, or similar sensor semiconductor fabrication. The Bayer filter is completely integrated within or affixed to the sensor and cannot be removed, replaced, or adjusted. When light impinges the surface of an RGB sensor, the underlying photosensors register a signal related to the intensity of the impinging wavelengths, as a function of the color of the integrated sensor, directly over each photosensor device. The disclosed technology does not require expensive special cameras, filter wheels, complex optical alignments, or stationary, non-handheld components.

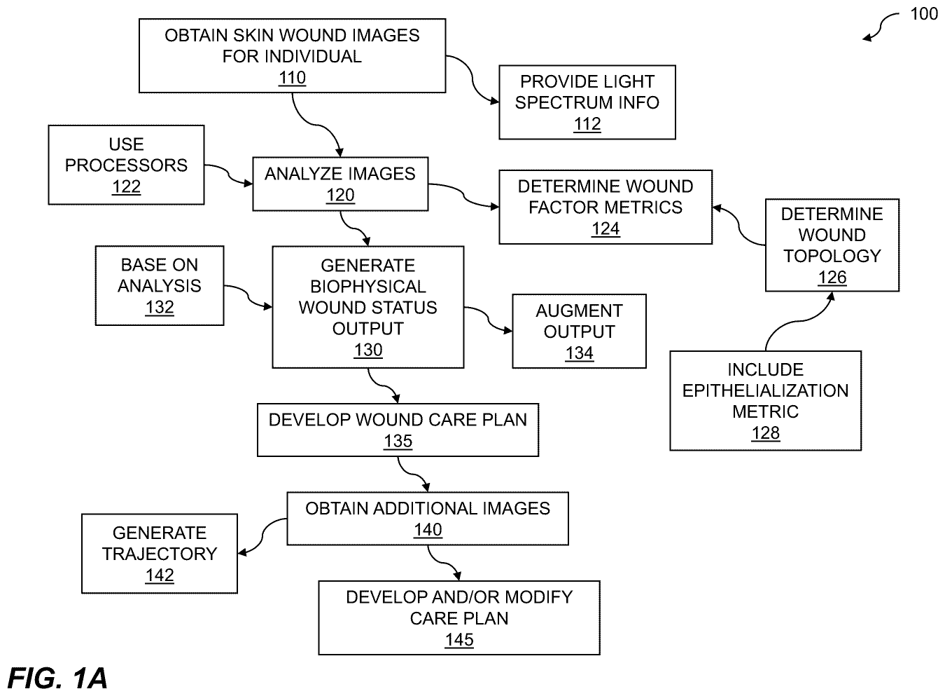

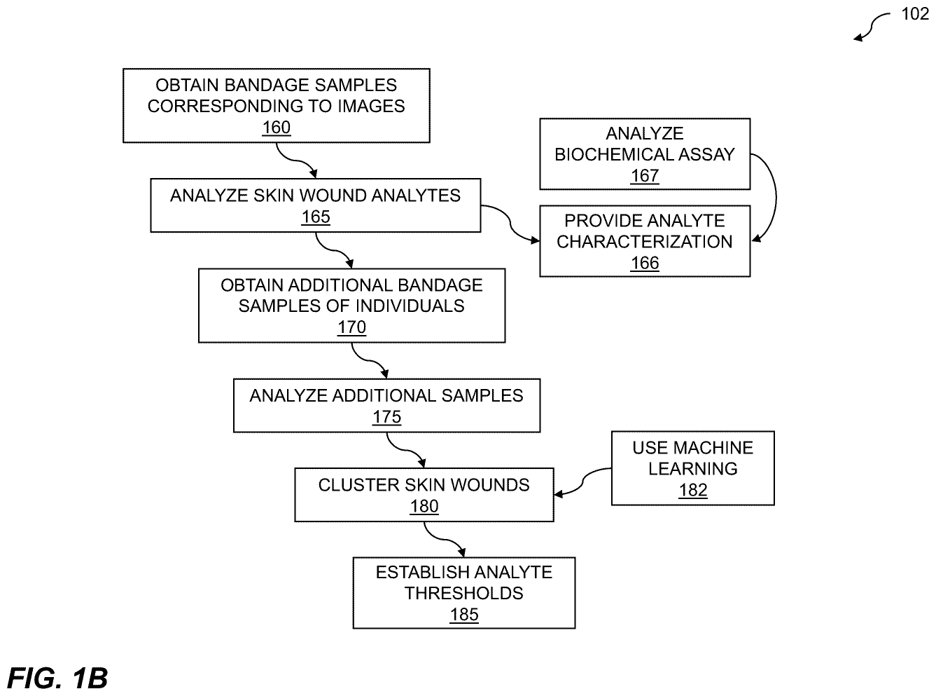

FIG. 1A is a flow diagram for image analysis using skin wound factors. Determination of the biophysical status of a material sample such as a skin wound can be enabled by image analysis of skin wound images. One or more skin wound images are obtained for an individual. The skin wound images provide light spectrum information that includes fluorescence characteristics and reflectance characteristics. The light spectrum information can further include infrared thermal characteristics. One or more processors are used to analyze the skin wound images to determine metrics for skin wound factors, based on the light spectrum information. The skin wound factors comprise infection, inflammation, granulation, topology, and epithelialization. An output that is indicative of biophysical skin wound status is generated. The output is based on the analyzing of the skin wound factors.

The flow 100 includes obtaining 110 one or more skin wound images for an individual. The one or more skin wound images can include high-resolution images, low-resolution images, and so on. The images can include black and white images, grayscale images, color images, false-color images, etc. The images can include contemporaneous images; images taken over a period of time such as days, weeks, or months; and the like. In embodiments, the images can include images captured by one or more Red-Green-Blue (RGB) sensors. The skin wound images can be based on illuminating, exciting, irradiating, etc. the skin wound with various light spectra. In embodiments, the illumination or excitation light wavelength can be located within the visible spectrum and/or outside of the visible spectrum.

In the flow 100, the skin wound images provide 112 light spectrum information that includes fluorescence characteristics and reflectance characteristics. The fluorescence characteristics can include absorbing light of one wavelength and emitting light at a second wavelength. The wavelength of the light that is absorbed can be shorter than the wavelength of the light that is emitted or fluoresced. The skin wound images can include fluorescence characteristics along the Red-Green-Blue (RGB) light wavelength spectrum. The reflectance characteristics can include reflecting light at a wavelength. The skin wound images can include reflectance characteristics along the Red-Green-Blue (RGB) light wavelength spectrum. The skin wound images can include a data point or data points related to the fluorescence characteristics and reflectance characteristics of the skin wound of the material sample. The fluorescence characteristics and the reflectance characteristics of the skin wound can be ascertained using a component such as an RGB sensor. Each output red, green, and blue of the RGB sensor provides a relative measurement of the skin wound fluorescence and reflectance while the skin wound is irradiated with the excitation light wavelength. Data associated with the RGB sensor can be captured within the one or more skin wound images. In embodiments, the light wavelength that excites fluorescence characteristics and reflectance characteristics of the material sample can be substantially a 395 nm light wavelength. A wavelength can be said to be substantially at a certain wavelength when its spectral energy peak encompasses that wavelength within 10% of the wavelength.

The flow 100 includes analyzing 120 the skin wound images. The analyzing the images can be based on applying one or more of image processing techniques, skin wound analysis techniques, and so on. The image processing techniques that can be applied to skin wound analysis can include edge enhancement, contrast enhancement, dithering and half-tone tasks, feature detection, and so on. In the flow 100, the analyzing uses 122 one or more processors. The one or more processors can be associated with a handheld device such as a smartphone, tablet, or specialized device; a computing device such as a desktop computer or a laptop computer; a server device such as a local server, a network-accessible server, a cloud-based service (e.g., computing as a service “CaaS”); and so on. The one or more processors can be used to perform one or more image processing tasks, algorithms, heuristics, processes, etc. In the flow 100, the analyzing determines metrics 124 for skin wound factors, based on the light spectrum information. The metrics can be based on a count or value; a range of values; a percentage; a qualitative evaluation such as highly likely, likely, unlikely, and highly unlikely; and so on. The metrics can include “present” or “not present”. Various skin wound factors can be determined. In embodiments, the skin wound factors comprise infection, inflammation, granulation, topology, and epithelialization. In embodiments, the analyzing can be based on a subset of the skin wound factors. The factors can be further based on physical characteristics, chemical characteristics, and so on. In embodiments, the metrics can include physical and chemical skin wound signatures. The signatures can be associated with excitation, illumination, or irradiation light spectra.

In the flow 100, the analyzing determines 126 wound topology. Various characteristics associated with the wound can be analyzed for wound topology. In embodiments, the wound topology can include wound area, depth, profile, texture, roughness, shape, edge definition, and so on. Other skin wound characteristics for wound topology can be considered. In other embodiments, the wound topology can be identified based on a measure of granulation, slough, eschar, flavin adenine dinucleotide (FAD), and/or nicotinamide adenine dinucleotide plus hydrogen (NADH) in the wound sample. The wound topology can be based on further factors, metrics, and so on. The determining wound topology can include an epithelialization metric 128. In embodiments, an epithelialization metric can be included in determining skin wound topology. Epithelialization can be based on the presence of epithelial cells. Epithelial cells can include surface cells associated with tissue such as human body tissue including skin cells, blood vessel cells, and organ cells. Epithelialization includes a biological process for covering a denuded epithelial surface such as the surface of the skin. The denuded epithelial surface of the skin can result from an abrasion, a laceration, a puncture, surgery, etc. In embodiments, the epithelialization can be identified based on a measure of nicotinamide adenine dinucleotide (NAD), NADH, and/or FAD.

Recall that epithelialization is the final step in the closing of a wound. By way of analogy, the earlier parts of wound healing can be compared to rebuilding a house foundation and putting up the framing; epithelialization is analogous to putting on the roof. The epithelial tissue does that by cell multiplication at the edge of the wound which then, in effect, “crawls” over the wound bed. To the naked eye, epithelization is essentially invisible, because it is only a cell or two thick. The disclosed epithelialization metric is based on detecting the cellular activity of the healing wound's keratinocyte cells that are proliferating and moving at the wound edge. This is accomplished by monitoring NADH activity using the detection of blue light fluorescence emissions (˜495 nm and ˜525 nm) resulting from the impingement of ultraviolet illuminations (˜280 nm and ˜325 nm) on the wound region. The cellular activity indicated by NADH activity biologically, rather than visually, defines the location of the wound edge. By using the “blue halo” metric to biologically and spatially define the wound edge, the area of the wound can be more precisely measured and boundaries can be defined for the inbounds area of the wound, which can be used for additional calculations on other wound metrics. Clinically, the biologically-defined wound edge can warn clinicians not to disturb the mostly invisible epithelial tissue and critically avoid cleaning or debriding the wound where it has already, but invisibly, healed. Thus, in embodiments, the epithelialization metric is based on detecting a nicotinamide adenine dinucleotide plus hydrogen (NADH) spatial halo around the skin wound.

The flow 100 includes generating 130 an output indicative of biophysical skin wound status. The biophysical status of a skin wound can be used to gauge, ascertain, track, compare, and so on a status of the skin wound. The status can be based on the presence or absence of one or more analytes, the presence or absence of infection, healing progress, wound care plan efficacy, and so on. The biophysical status can be based on the presence or absence of cells within skin wound exudate. In embodiments, the biophysical status can enable a skin wound assessment. The skin wound assessment can be critical to determining a stage of wound healing, the efficacy of a wound treatment plan, and the like. Such skin wound assessment can be particularly critical in tracking wound healing of chronic wounds. The chronic wounds can result from diseases such as diabetes, pressure ulcers caused by pressure to the skin, etc. In embodiments, the skin wound assessment can be performed longitudinally. The longitudinal assessment can include skin wound assessment over a period of time. The period of time can include one or more hours, days, weeks, months, etc.

In the flow 100, the output is based on the analyzing 132 of the skin wound factors. The analysis can be performed on one or more skin wound images for an individual that were obtained at substantially the same time. The analysis can be performed on one or more skin wound images obtained longitudinally over a period of time. The analysis can be based on one or more image processing techniques, where the image processing techniques can be applied to the one or more skin wound images obtained for the individual. The one or more images can be based on data obtained from one or more sensors such as RGB sensors. The analysis can include a multi-factor analysis. Described below, the analysis can be based on five-factor analysis. In embodiments, the analysis can include identifying a wound topology within the material sample. The wound topology can include wound length and wound width, wound area, wound contour, wound depth, wound volume, wound texture, and so on. In embodiments, the wound topology can be identified based on a measure of granulation, slough, eschar, flavin adenine dinucleotide (FAD), and/or nicotinamide adenine dinucleotide plus hydrogen (NADH) in the wound sample. In embodiments, the analysis can include identifying inflammation within the material sample. Inflammation can include a normal, initial stage of wound healing. Indications of inflammation associated with a skin wound can include heat and redness, pain or tenderness of the wound and surrounding tissue, elevated body temperature or fever, etc. In embodiments, the inflammation that is determined by the analysis can be identified based on a measure of heat, redness, and swelling in the material sample.

In embodiments, the analysis can include identifying epithelialization within the material sample. Epithelialization can be based on the presence of epithelial cells. Epithelial cells can include surface cells associated with tissue such as human body tissue including skin cells, blood vessel cells, and organ cells. Epithelialization includes a biological process for covering a denuded epithelial surface such as the surface of the skin. The denuded epithelial surface of the skin can result from an abrasion, a laceration, a puncture, surgery, etc. In embodiments, the epithelialization can be identified based on a measure of nicotinamide adenine dinucleotide (NAD), NADH, and/or FAD. In embodiments, the analysis can include identifying granulation within the material sample. Granulation within a wound can be associated with a wound healing process. Granulation tissue can include a lumpy, pink tissue that can contain connective tissue and capillaries. Granulation tissue can grow from an edge of a wound toward the center of the wound. In embodiments, the granulation can be identified based on a measure of hemoglobin and collagen.

In embodiments, the analysis can include identifying infection within the material sample. An infection can be based on the presence of undesirable bacteria, fungi, microorganisms, viruses, yeast, etc. An infection can first occur in a portion of a body and can spread to another portion of the body. An infection can include a chronic infection. The infection can destroy tissue thereby hindering wound healing, worsening a wound status, etc. An infection can present as an elevated body temperature, suppuration, etc. In embodiments, the infection can be identified based on a measure of a porphyrin, pyoverdine, slough, eschar, or an inflammation signature. The measure of porphyrin, pyoverdine, slough, eschar, or inflammation can be determined based on the fluorescence characteristics and the reflectance characteristics of the skin wound sample, by examining the wound exudate, etc. In embodiments, the inflammation signature can include wound temperature, wound water content, and periwound redness. The wound temperature can be based on capturing a thermal image of the skin wound. The wound water content can be determined by analyzing the fluorescence characteristics and the reflectance characteristics of the skin wound. In embodiments, identifying wound topology, inflammation, epithelialization, granulation, and infection comprises a five-factor biophysical material sample status.

The one or more skin wound images obtained for an individual can provide light spectrum information. The light spectrum information that has been discussed so far can include fluorescence characteristics and reflectance characteristics. Other characteristics associated with light spectrum information can also be provided within the one or more images. In embodiments, the light spectrum information can include infrared thermal characteristics. The infrared thermal characteristics can include thermal characteristics of the skin wound, surrounding tissue, the body of the individual, and so on. The thermal characteristics can include a thermal profile of the skin wound. The thermal characteristics can indicate infection, wound irritation, fever, etc. The flow 100 further includes augmenting 134 the output, based on analysis of the infrared thermal characteristics. The augmenting the output can include providing further information about a skin wound to a healthcare provider, more accurate information, etc. The generated output and the augmented output can be used to determine a wound care plan, a healing trajectory, etc.

The flow 100 further includes developing 135 a skin wound care plan, based on the output. The wound care plan can include one or more therapies such as drug therapy, physical therapy, and so on. The one or more therapies can supplement or augment other aspects of the skin wound care plan. In embodiments, the skin wound care plan can include a cleansing protocol or recommendation, a type of wound dressing, a frequency of dressing change, etc. The flow 100 further includes obtaining 140 additional skin wound images for the individual. The additional skin wound images can include substantially similar light spectrum information to the skin wound images, substantially different light spectrum information, a combination of light spectra information, and so on. The additional skin wound images can be obtained at substantially the same time as the skin wound images, can be obtained at one or more later times, and the like. The flow 100 further includes generating 142 a wound healing trajectory, based on the one or more skin wound images and at least one additional skin wound image. Skin wound healing can be thought to comprise four stages: hemostasis, inflammatory, proliferation, and maturation. The wound healing trajectory that can be generated can indicate the progress of a wound through the stages of healing.

Obtaining skin wound images and additional skin wound images can enable skin wound assessment to be performed longitudinally. The longitudinal assessment can be based on a wound care treatment plan. The treatment plan can include drug therapy, bandage type and bandage change frequency, surgery, etc. In embodiments, the additional skin wound images can provide a time-sequenced representation of skin wound healing. The skin assessment performed longitudinally can enable development of a wound healing trajectory. The flow 100 further includes developing and/or modifying 145 a skin wound care plan, based on an analysis of the wound healing trajectory. A wound healing trajectory can be based on stages of healing. The stages of wound healing associated with a healing trajectory can include stages such as hemostasis, inflammatory, proliferation, and maturation stages. In further embodiments, the wound healing trajectory can be used to modify a wound care treatment plan. In addition to capturing and analyzing the fluorescence characteristics, the reflectance characteristics, and the thermal characteristics associated with the skin wound images, other assay techniques can be applied to the skin wound sample. In embodiments, the skin wound sample can be analyzed using a biochemical assay of wound analytes. Discussed below, the biochemical assay of wound analytes can be accomplished using a lateral flow assay (LFA) technique. The wound analytes can be obtained from wound exudate, where the wound exudate can be collected from a bandage covering a wound, a wound drainage tube, directly from the wound bed, etc. In embodiments, the output for a material sample comprising the biochemical assay of wound analytes can be analyzed in conjunction with an output for a material sample comprising a skin wound.

Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

FIG. 1B is a flow diagram for bandage sampling. Bandage sampling can include extracting one or more exudate samples from one or more bandages associated with an individual. The exudate samples can also be collected from a skin wound bed. Further exudate samples can be collected from one or more bandages associated with one or more further individuals. The exudate samples correspond to skin wounds within skin wound images for an individual. The examples can be analyzed for analytes associated with a skin wound. The analysis of the analytes can be used to track wound healing progress, to develop a wound care plan, and so on. Bandage sampling supports image analysis using skin wound factors. One or more skin wound images for an individual are obtained, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors. The light spectrum information can include infrared thermal characteristics. The generated output that is indicative of biophysical skin wound status can be augmented, based on analysis of the infrared thermal characteristics. The analysis of the skin wound images and the analysis of the infrared thermal characteristics are used to identify biophysical skin wound status. The identifying includes identifying wound infection, inflammation, granulation, topology, and epithelialization. The identifying infection, inflammation, granulation, topology, and epithelialization comprises a five-factor biophysical material sample status.

Wound healing is a principal concern of healthcare professionals who provide wound treatment to patients. A wound, which can include a break in skin or other body tissues associated with the patient, can result from surgery, injuries, trauma injuries, disease, and so on. The healing of a wound depends on a variety of factors. The healing factors can include desiccation, infection, or unusual bacteria presence in a wound; the age of the patient since older patients tend to heal more slowly; patient body type such as thin, average, overweight, or obese since obese patients can experience poor blood circulation in adipose tissue; patient nutrition since poor nutrition can hinder healing; and other medical conditions or treatments such as an immunosuppressed patient, a patient undergoing chemotherapy or immunotherapy treatments, etc. Further, wound healing can be better characterized and enhanced by understanding what is “going on” within the wound. A visual inspection of a wound can yield some information about the state of the wound, healing progress, and so on. A visual inspection of the wound can observe swelling or redness; an examination of bandages removed from covering a wound can observe discharge associated with bleeding or suppuration; etc. However, visual examination of the wound only enables determination of a broad status of the wound rather than a detailed review of what healing-related factors are present, or what processes are occurring within the wound. In order to determine what is going on within a wound, a generated material sample status can be based on a five-factor biophysical status. The five-factor biophysical status can include identifying wound topology, inflammation, epithelialization, granulation, and infection. These five factors can be identified based on analysis of fluorescence characteristics and reflectance characteristics of the material sample. The material sample can include the skin wound.

Another technique that can be used to determine a biophysical status of a wound such as a skin wound is based on a lateral flow assay (LFA). A lateral flow assay is used to detect the presence or absence of a target analyte. The target analyte can include a pathogen, a biomarker, and so on within a sample. The LFA can be used to detect target analytes in samples obtained from humans and animals. The LFA can also be used to detect contaminants within water supplies and foodstuffs for humans and animals. The LFA techniques are also used to detect diseases such as COVID-19 and are commonly used for in-home tests such as pregnancy tests. The LFA techniques discussed herein can be based on immunoassay techniques. An immunoassay technique can use a membrane such as a nitrocellulose membrane to which a sample can be supplied. The sample can include a liquid, where the liquid can include a complex liquid. In addition to the nitrocellulose membrane, the LFA can include nanoparticles or other particles or molecules that have been dyed or colored, and antibodies that target specific analytes, and can be characterized visually or optically to provide qualitative, semi-quantitative, or quantitative results. The LFAs may include one or more antibodies that target one or more regions of the analyte in a sandwich or competitive scheme. The LFA may also include a sample pad which includes chemical additives to increase the performance of the immunoassay. The LFA typically includes a “control” that can be used to indicate that the test is working as expected. The control can be indicated by a color bar on the nitrocellulose membrane. The presence of analytes within the test sample can also be indicated by a color bar on the nitrocellulose membrane. The biophysical result is based on analysis of the fluorescence characteristics and the reflectance characteristics associated with the material sample.

Identification of water content within a wound such as a skin wound further assists in determining biophysical status of the skin wound. The water content can be linked to an inflammation signature associated with a wound. The inflammation signature can be associated with the status of wound healing. Unlike most biochromes, water exhibits a monotonically increasing absorption characteristic across an excitation profile as the excitation wavelength increases. Because water is such an integral component of living tissue, water identification can be very useful. Determining the water content within a wound can be accomplished by monitoring light wavelength absorption at 800 nm to 1000 nm and comparing the light wavelength absorption to absorption at shorter wavelengths. The absorption can be determined based on a reduction in reflectance as detected by a sensor such as a Red-Green-Blue (RGB) sensor. Absorption by most chromophores found in nature decreases with increasing wavelength; however, in the case of water, the opposite is true. A light source with peak intensity from 800 nm to 1000 nm can be used to generate an absorption signal based on a comparison to a diffuse reflectance standard:

Absorption Image=−log((Raw Image−Dark Image)/Exp/(DR Image−Dark Image)/Exp)

where Exp is the exposure time, the Raw Image is the output of the RGB sensor with excitation light illumination as described herein, the Dark Image is the output of the RGB sensor with no excitation light illumination and only ambient lighting conditions, and the DR (diffuse reflectance) Image standard is a known and characterized sample that provides a baseline output of the RGB sensor with excitation light illumination.

The flow 102 includes obtaining bandage samples 160 corresponding to the skin wound images. The bandage samples can be obtained when a bandage is removed to check a skin wound, as part of a bandage change protocol based on a skin wound care plan, and so on. One or more exudate samples can be extracted from a single bandage sample, a plurality of bandage samples, and so on. In other embodiments, the one or more exudate samples can be obtained from the wound bed of the skin wound. The one or more exudate samples can include water, electrolytes, inflammatory mediators, nutrients, white cells, enzymes that digest proteins, and growth factors. In embodiments, the bandage samples can contain skin wound analytes. The exudate samples can be analyzed for the presence or absence of one or more analytes. The flow 102 includes analyzing 165 the skin wound analytes. In embodiments, the analyzing can be accomplished using one or more processors. The processors can include one or more processors within a handheld device such as a smartphone, tablet, or specialized device; a desktop or laptop computer; a server; and so on. In the flow 102, the analyzing is performed to provide an analyte characterization 166. The characterization of skin wound analytes can include determining whether an analyte of interest is present or not, determining the concentration of an analyte, and so on. In embodiments, the analyte characterization can provide skin wound biochemical information. Discussed previously, an output indicative of biophysical skin wound status can be generated, wherein the output is based on the analyzing of the skin wound factors. In embodiments, the analyte characterization can be used to augment the output that was generated.

The flow 102 includes analyzing the skin wound analytes to provide an analyte characterization 166. The skin wound analytes can be analyzed using one or more assay technique. In embodiments, the analyte characterization is accomplished by analyzing a biochemical assay 167. The wound analytes can be obtained from wound exudate, where the wound exudate can be collected from a bandage covering a wound, a wound drainage tube, directly from the wound bed, etc. In other embodiments, the biochemical assay can include an immunochromatographic assay. An immunochromatographic assay can be based on an immunoaffinity partition of an analyte between a sample migrating laterally through a porous membrane and a finite portion or affinity zone. The affinity zone can be based on immobilizing an antibody on a portion of the porous membrane. In embodiments, the biochemical assay can include an immunosorbent assay. The immunosorbent assay can be based on the immobilized antibody. Discussed below, the biochemical assay of skin wound analytes can be accomplished using an assay technique such as a lateral flow assay (LFA) technique. In embodiments, the analyte characterization is used to augment a generated output. The generated output can be indicative of biophysical skin wound status, where the output is based on the analyzing of the skin wound factors. In embodiments, analyzing the skin wound analytes within the bandage sample can be based on a biochemical assay of skin wound analytes.

The flow 102 further includes obtaining additional bandage samples 170 which correspond to skin wounds on a plurality of individuals. One or more additional bandage samples can be obtained from the plurality of individuals. The first individual and the plurality of individuals can include patients with substantially similar wounds, injuries, infections, diseases, and so on. The additional bandage samples can be obtained to compare or contrast healing rates among patients, efficacy of different wound care plans, etc. The flow 102 further includes analyzing the additional bandage samples 175 to provide additional analyte characterization. The additional analyte characterizations can be based on one or more metrics for skin wound factors, where the skin wound factors can include infection, inflammation, granulation, topology, epithelialization, and so on.

Infection can include an infection based on bacteria, fungi, viruses, yeast, microorganisms, and so on. An infection can occur in a portion of a body and can spread to another portion of the body. The infection can hinder the healing of a wound. If left untreated, an infection can threaten the life of an individual. An infection can present as an elevated temperature, suppuration, etc. Inflammation can indicate an initial or first stage of wound healing. The inflammation can include heat and redness of the wound, pain or tenderness of the wound and surrounding tissue, elevated body temperature or fever, and so on. In embodiments, the inflammation that is determined by the analysis can be identified based on a measure of heat, redness, and swelling in the material sample. Granulation tissue can include a pink tissue that can contain connective tissue and capillaries. Granulation tissue can grow from an edge of a wound toward the center of the wound. In embodiments, the granulation can be identified based on a measure of hemoglobin and collagen. The wound topology can include wound area, volume, depth, profile, texture, roughness, and so on. In embodiments, the wound topology can be identified based on a measure of granulation, slough, eschar, flavin adenine dinucleotide (FAD), and/or nicotinamide adenine dinucleotide plus hydrogen (NADH) in the wound sample. Epithelial cells, such as skin cells, blood vessel cells, and organ cells, can include surface cells associated with a human body. Epithelialization can include a biological process for covering a denuded epithelial surface such as the surface of the skin. The denuded epithelial surface of the skin can result from an abrasion, a laceration, etc. In embodiments, the epithelialization can be identified based on a measure of nicotinamide adenine dinucleotide (NAD), NADH, and/or FAD.

The flow 102 further includes clustering the skin wounds 180 on the plurality of individuals. The clustering can be performed manually by personnel such as trained experts, by processing, and so on. The clustering can be based on statistical analysis. The clustering can be based on types of skin wounds, location, severity, cause, and so on. The cause of a skin wound can include injury, surgery, disease, and the like. In the flow 102, the clustering is performed using machine learning 182. The machine learning can be accomplished by a trained network such as a trained neural network. The training can be accomplished by providing a dataset such as a training dataset with associated expected answers or inferences. As the training dataset is processed by the neural network that is being trained, weights associated with nodes or “neurons” within the neural network can be adjusted to improve inference accuracy and convergence speed. The flow 102 includes establishing analyte thresholds 185 for inflammation, infection, granulation, and epithelialization, based on the additional analyte characterization. The analyte thresholds can include one or more of a value; a range; a percentage; a qualitative assessment such as poor, fair, good, and excellent; and so on. The thresholds can include healing milestones. The thresholds can be used to gauge skin wound healing progress, to adjust a skin wound care plan, etc.

Various steps in the flow 102 may be changed in order, repeated, omitted, or the like without departing from the disclosed concepts. Various embodiments of the flow 102 can be included in a computer program product embodied in a non-transitory computer readable medium that includes code executable by one or more processors.

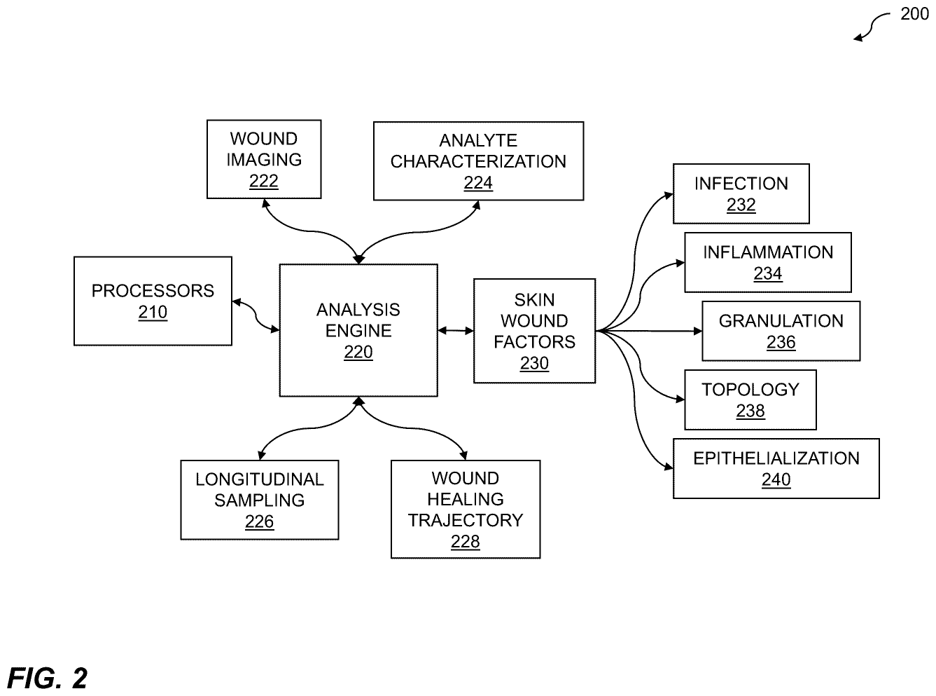

FIG. 2 is a high-level block diagram for five-factor analysis. Discussed above and throughout, determination of the status of a skin wound, such as a status of healing of the wound, can be difficult to evaluate. To improve accuracy of skin wound status determination, a five-factor analysis technique can be applied. The five-factor technique can be used to determine what is “going on” with the skin wound. The five-factor analysis can be used as a basis for generating an output indicative of biophysical skin wound status. The output status can be used to develop a wound care plan, to track wound healing, and so on. The five-factor analysis can be based on applying image processing techniques to enhance the analyzing of skin wound images. The five-factor analysis enables skin wound image analysis using skin wound factors. The five-factor biophysical status can include identifying wound topology, inflammation, epithelialization, granulation, and infection. These five factors can be identified based on analysis of fluorescence characteristics and reflectance characteristics within images of a skin wound. One or more skin wound images for an individual are obtained, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors. A thermal image of the material sample is further captured. The generated output is augmented based on an analysis of the thermal image. The output is augmented, based on analysis of the infrared thermal characteristics.

The system block diagram 200 includes one or more processors 210. The processors can include one or more standalone processors, processors associated with devices such as handheld devices such as smartphones or tablets, special-purpose devices, and so on. The processors can be associated with computing devices such as desktop and laptop computers, local servers, blade servers, Internet-connected servers, cloud servers, etc. The processors can include general purpose processors, processor cores within field-programmable gate arrays (FPGAs) and application-specific integrated circuits (ASICs), custom integrated circuits or chips, and so on. The system block diagram can include an analysis engine 220. The analysis engine can comprise hardware, software, firmware, etc., that can be used to execute image processing techniques, wound analysis techniques, lateral flow analysis (LPA) techniques, a skin wound treatment care plan, and the like. In the block diagram 200, the analysis engine can be coupled to a wound imaging 222 component. In embodiments, the wound imaging component can obtain one or more skin wound images for an individual. The one or more skin wound images can result from sensing light spectrum information resulting from illuminating or irradiating the skin wound. The light spectrum information can include infrared light, visible light, and other electromagnetic spectra. The sensing the light spectrum information can be accomplished using an infrared sensor, a Red-Green-Blue (RGB) sensor, etc. The skin wound images provide light spectrum information that includes fluorescence characteristics and reflectance characteristics.

In the block diagram 200, the analysis engine can be coupled to an analyte characterization 224 component. The skin wound analytes can be obtained using a variety of techniques. Embodiments further include obtaining bandage samples corresponding to the skin wound images. The bandages can include one or more liquids, such as liquid exudate, associated with the skin wound. In embodiments, the bandage samples can contain skin wound analytes. One or more analytes associated with a skin wound can be obtained from one or more bandages, from a wound bed of a skin wound, and so on. Discussed below, a biochemical assay of wound analytes can be accomplished using a lateral flow assay (LFA) technique. The wound analytes can be obtained from wound exudate, where the wound exudate can be collected from a bandage covering a wound, a wound drainage tube, directly from the wound bed, etc. In embodiments, the output for a material sample comprising the biochemical assay of wound analytes can be analyzed in conjunction with a generated biophysical skin wound status (discussed below). Further embodiments can include analyzing the skin wound analytes to provide an analyte characterization. The analytes can indicate infection, presence or absence of cells associated with wound healing, etc. Embodiments include analyzing the skin wound analytes to provide an analyte characterization. The analytes can be characterized based on analysis of more than one exudate sample. Further embodiments can include obtaining additional bandage samples corresponding to skin wounds on a plurality of individuals. The additional bandage samples can be used to compare analyte characterizations for similarities or differences between skin wounds associated with the plurality of individuals, skin wound types such as ulcers associated with diseases, etc. The analysis of the additional samples can provide additional analyte characterization.

In the block diagram 200, the analysis engine can be coupled to a longitudinal sampling component 226. The longitudinal sampling component can enable longitudinal assessment of a skin wound over a period of time. The longitudinal assessment can be used to track skin wound status changes, to track healing of the skin wound, to quickly determine a lack of or slow healing, and so on. In embodiments, the skin assessment performed longitudinally can enable a wound care treatment plan. The treatment plan can include drug therapy, bandage type and bandage change frequency, surgery, etc. In embodiments, the skin wound sample can include a biochemical assay of wound analytes. Discussed below, the biochemical assay of wound analytes can be accomplished using a lateral flow assay (LFA) technique. The wound analytes can be obtained from wound exudate, where the wound exudate can be collected from a bandage covering a wound, a wound drainage tube, directly from the wound bed, etc. In the block diagram 200, the analysis engine can be coupled to a wound healing trajectory component 228. The wound healing trajectory component can determine a skin wound status with respect to four stages of wound healing. The four stages of wound healing can include hemostasis, inflammatory, proliferative, and maturation. In other embodiments, the skin wound assessment performed longitudinally can enable development of a wound healing trajectory. A wound healing trajectory can be based on the four stages of healing. In further embodiments, the wound healing trajectory can be used to modify a wound care treatment plan. In addition to capturing and analyzing the fluorescence characteristics and the reflectance characteristics associated with the skin wound images, other assay techniques can be applied to the skin wound sample.

The system block diagram 200 can include a skin wound factors component 230. The skin wound factors can be based on five factors comprising a five-factor biophysical status of the skin wound. The five-factor biophysical status can include identifying infection 232, inflammation 234, granulation 236, topology 238, and epithelialization 240. These five factors can be identified based on analysis of fluorescence characteristics and reflectance characteristics of the skin wound images. The five factors associated with determining the biophysical status of the skin wound can include infection 232. An infection can be based on the presence of undesirable bacteria, fungi, microorganisms, viruses, yeast, etc. An infection can first occur in a portion of a body and can spread to another portion of the body. An infection can include a chronic infection. The infection can destroy tissue thereby hindering wound healing, worsening a wound status, etc. An infection can present as an elevated body temperature, suppuration, etc. In embodiments, the infection can be identified based on a measure of a porphyrin, pyoverdine, slough, eschar, or an inflammation signature. The measure of porphyrin, pyoverdine, slough, eschar, or inflammation can be determined based on of the fluorescence characteristics and the reflectance characteristics of the skin wound sample, by examining the wound exudate, etc.

The five factors associated with determining the biophysical status of the skin wound can include inflammation 234. Inflammation can include a normal, initial stage of wound healing. Inflammation associated with the skin wound can include a variety of wound characteristics such as heat and redness, pain or tenderness of the wound and surrounding tissue, elevated body temperature or fever, etc. In embodiments, the inflammation that is determined by the analysis can be identified based on a measure of heat, redness, and swelling indicated by image analysis of the obtained skin wound images for the individual. The five factors associated with determining the biophysical status of the skin wound can include granulation 236. Wound granulation is part of wound healing where granulation tissue matrix can fill the wound as part of skin rebuilding. Granulation tissue can include a lumpy, pink tissue that can contain connective tissue and capillaries. Granulation tissue can grow from an edge of a wound toward the center of the wound. In embodiments, the granulation can be identified based on a measure of hemoglobin and collagen.

The five factors associated with determining the biophysical status of the skin wound can include wound topology 238. Wound topology can be used to describe a type of wound such as an abrasion, laceration, puncture, or avulsion wound; to describe wound edges such as diffuse or rolled, etc. In embodiments, the wound topology includes wound area, volume, depth, profile, texture, roughness, and so on. The wound topology can be determined based on image processing of the obtained skin wound images. In further embodiments, the wound topology can be identified based on a measure of granulation, slough, eschar, flavin adenine dinucleotide (FAD), and/or nicotinamide adenine dinucleotide plus hydrogen (NADH) in the wound sample. These latter measurements associated with wound topology can be determined using lateral flow assay, described previously and below. The five factors associated with determining the biophysical status of the skin wound can include epithelialization 240. Epithelialization can include a biological process that includes covering an abraded, denuded, etc. epithelial surface. The epithelialization can be identified based on a measure of nicotinamide adenine dinucleotide (NAD), NADH, and/or FAD. In embodiments, the measurements can be accomplished by image analysis, LFA, and so on.

The image analysis and other analyses that can be performed by the analysis engine can be supplemented by further analysis techniques. Another technique that can be used to determine the biophysical status of a wound such as a skin wound is based on a lateral flow assay (LFA). A lateral flow assay is used to detect the presence or absence of a target analyte. The target analyte can include a pathogen, a biomarker, and so on within a sample. The LFA can be used to detect target analytes in samples obtained from humans and animals. The LFA can also be used to detect contaminants within water supplies and foodstuffs for humans and animals. The LFA techniques are also used to detect diseases such as COVID-19 and are commonly used for in-home tests such as pregnancy tests. The LFA techniques discussed herein can be based on immunoassay techniques. An immunoassay technique can use a membrane such as a nitrocellulose membrane to which a sample can be supplied. The sample can include a liquid, where the liquid can include a complex liquid. In addition to the nitrocellulose membrane, the LFA can include nanoparticles or other particles or molecules that have been dyed or colored, and antibodies that target specific analytes and can be characterized visually or optically to provide qualitative, semi-quantitative, or quantitative results. The LFAs may include one or more antibodies that target one or more regions of the analyte in a sandwich or competitive scheme. The LFA may also include a sample pad which includes chemical additives to increase the performance of the immunoassay. The LFA typically includes a “control” that can be used to indicate that the test is working as expected. The control can be indicated by a color bar on the nitrocellulose membrane. The presence of analytes within the test sample can also be indicated by a color bar on the nitrocellulose membrane. The biophysical result is based on analysis of the fluorescence characteristics and the reflectance characteristics associated with the material sample.

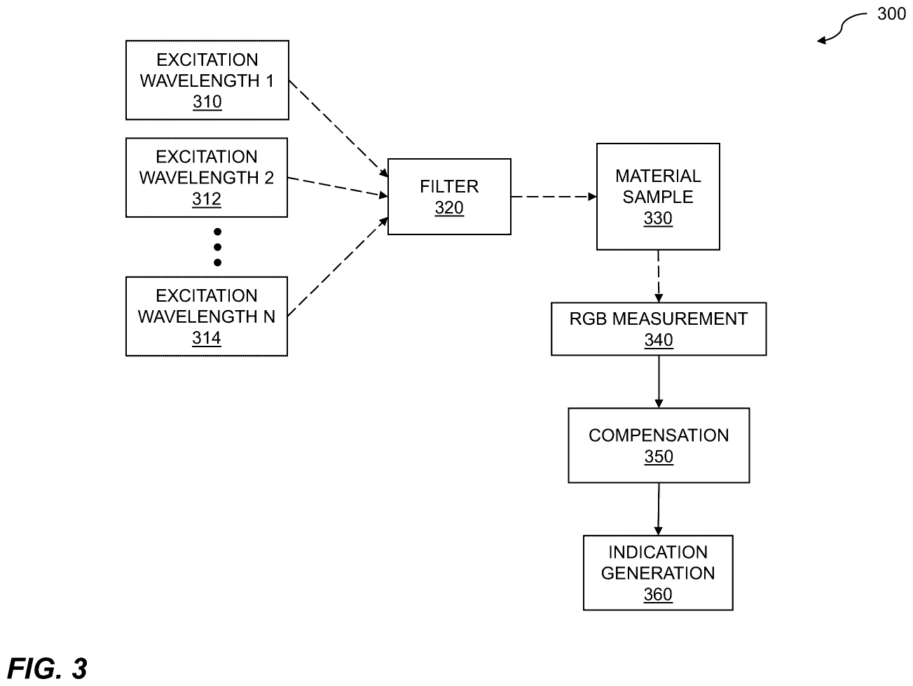

FIG. 3 shows a system block diagram for multispectral sample analysis. In the system block diagram 300, one or more absorption excitation light wavelengths are provided to a material sample, such as excitation wavelength 1 310, excitation wavelength 2 312, up to excitation wavelength N 314. The excitation wavelengths can emanate from a single source or from multiple sources, such as from an incandescent light source, an LED light source, a laser light source, an ultraviolet (UV) light source, an infrared (IR) light source, and so on. The excitation wavelengths can illuminate a sample, and the exciting enables capture of fluorescence characteristics of the material sample. The system block diagram illustrates multispectral sample analysis based on image analysis using skin wound factors. The skin wound factors are associated with wound care image analysis. One or more skin wound images for an individual are obtained, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors. A thermal image of the material sample is further captured. The generated output is augmented based on an analysis of the thermal image. The output is augmented, based on analysis of the infrared thermal characteristics.

The system block diagram 300 can include one or more optical filters 320 on the source side of a material sample 330. That is, the one or more excitation wavelengths 310, 312, and 314 can be conditioned by the one or more optical filters 320 such that the illuminating light from the excitation wavelengths is affected by the filters before it reaches the material sample 330. These filters do not affect the fluorescence emissions of the material sample that are detected by an RGB sensor, based on the illumination of the one or more excitation wavelengths. The filter 320 can be a bandpass filter. The one or more excitation wavelengths, as conditioned by any intervening filters 320, then impinge on a material sample 330, resulting in a fluorescence emission from the sample that is detected by RGB measurement block 340. Note that before RGB measurement block 340, the system block diagram 300 indicates light transmission, as denoted by the dashed lines among blocks 310, 312, 314, 320, 330, and 340. The output of RGB measurement block 340, as well as the signals between subsequent blocks 350 and 360, are electrical signals, as denoted by the solid lines. Optionally, an additional optical filter (not shown) can be placed between the material sample 330 and the RGB measurement 340. The additional optical filter can be a long-pass filter.

The electrical output of RGB measurement block 340 can be compensated by compensation block 350. Compensation can involve providing a boost or attenuation to electrical signals indicating a certain magnitude of a particular light wavelength in order to counteract sensor differences, ambient lighting differences, excitation wavelength spectra differences, and so on. The compensation block 350 can be adjusted based on various calibration techniques that are performed before or after an actual sample measurement. The output of compensation block 350 can enable generation of an indication 360 of a composition of a material sample. Analysis of the output of compensation block 350 (or directly from RGB measurement block 340) can enable generation of an indication of composition, based on the output of block 350 (or directly from block 340) using various methods such as table lookup, graph comparison, machine learning, human interpretation, signature comparison, and the like.

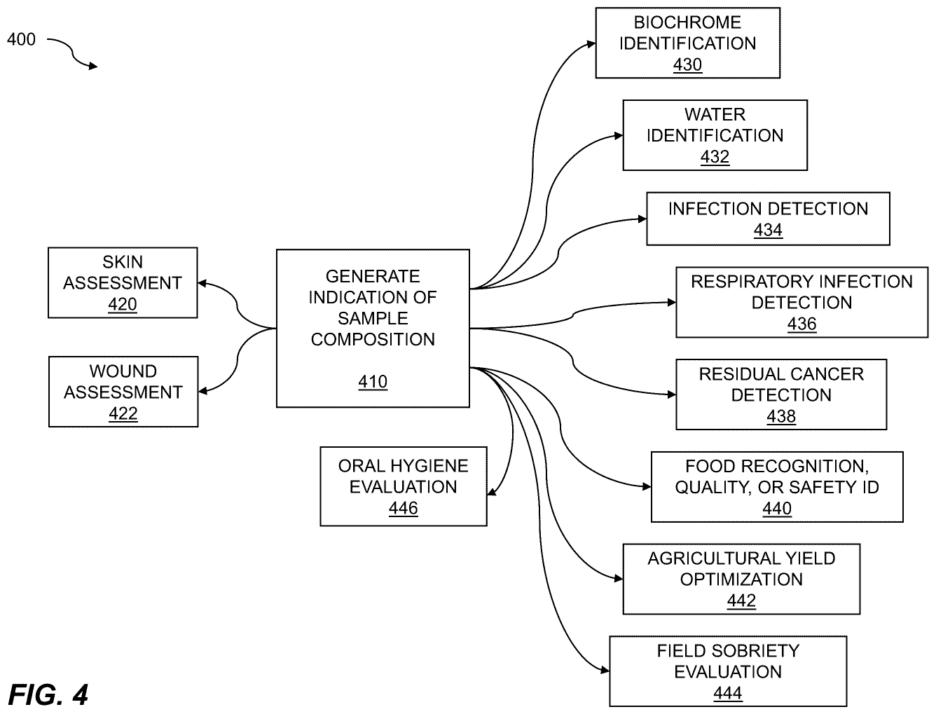

FIG. 4 shows a system block diagram for using fluorescence signatures. Fluorescence signatures can enable wound care image analysis using skin wound factors. The wound care image analysis can be applied to one or more skin wound images obtained for an individual. The skin wound images provide light spectrum information that includes fluorescence characteristics. The light spectrum information further includes reflectance characteristics. The wound image analysis is enabled by generating an output indicative of biophysical skin wound status. The one or more skin wound images can include a wound topology. An indication of biophysical skin wound status can be useful to a variety of human endeavors, as will be discussed below. The block diagram 400 can include generating an indication of sample composition 410. As discussed throughout, a light wavelength is excited on a material sample. The exciting enables capture of fluorescence characteristics of the material sample. The material sample exhibits fluorescence characteristics along the Red-Green-Blue (RGB) light wavelength spectrum. The material sample is illuminated with at least three additional light wavelengths. The illuminating enables capture of reflectance characteristics of the material sample. The material sample exhibits reflectance characteristics along the Red-Green-Blue (RGB) light wavelength spectrum. An output indicative of biophysical status of the material sample is generated. The output is based on analysis of the fluorescence characteristics and the reflectance characteristics. A thermal image of the material sample is further captured. The output is augmented based on an analysis of the thermal image.

The generated indication of biophysical status of the material sample can enable skin assessment 420. The skin assessment can involve predicting the onset of skin conditions such as psoriasis, which can be distinguished based on fluorescence from fluorophores such as melanin, elastin, collagen, keratin, and flavoprotein. Other skin conditions, such as eczema and acne, can also be predicted. In addition, skin hydration can be assessed using the disclosed techniques. The skin assessment can include feature identification. The indication can enable wound assessment 422. The wound assessment can be based on collecting a variety of images at different excitation wavelengths and spatially registering the images using micro-or macro-scale features, skin and wound edges, fiducial marks, reference standards for alignment, corresponding biological features, and the like. Feature recognition can be accomplished using Laplace of Gaussians, difference of Gaussians, Hessian-Laplace, scale invariant feature transform (SIFT), multi-scale-oriented patches (MOPS), or other image processing techniques for local feature description. Once corresponding features on images are identified, the registration technique can use translation, rigid body, rotation, or affine transformation methods to register multiple images collected at different wavelengths. A pixel-by-pixel registration allows for the images to be digitally processed in order to identify biological features, to perform calculations which isolate or enhance the biological signals, and/or to assess wound healing. Further analysis can enable algorithmic identification of infection. In embodiments, the wound assessment includes infection detection. In embodiments, the skin assessment includes wound assessment. In embodiments, the wound assessment is taken over time. In embodiments, the wound assessment is repeated over time and enables a wound care treatment plan. In embodiments, the skin assessment is updated using temporal change feature matching, that is, by comparing identified features in the wound to determine how they are changing temporally (i.e., with the passage of time). The temporal change can occur over two or more healthcare clinical sessions. At least one of the two or more healthcare clinical sessions can be self-administered.

As discussed previously, the indication can enable biochrome identification 430 and water identification 432. In addition, the indication can enable infection detection 434 or respiratory infection detection 436. Host metabolism plays a vital role in viral infections. Energy yielding metabolic pathways are repurposed by the virus to support viral replication. High concentrations of nicotinamide adenine dinucleotide+hydrogen (NADH) and flavins are indicative of such infections. The indication can be generated by isolating signals from NADH and flavins by collecting fluorescence photons in the R, G, and B channels, respectively, and exciting at or near 400 nm. This approach further isolates features in an image that can be attributed to the presence of flavins and NADH by taking the normalized ratio, where normalization is based on excitation flux, integration time, and channel sensitivity of the green channel signal to the blue channel signal, and isolating based on pixels that yield a ratio value indicative of the presence of NADH and/or flavins.

In addition, abnormal concentrations of porphyrin, which can be detected using the disclosed concepts, have been observed in serum from COVID-19 patients. Other respiratory-related infections, such as sinusitis, are more prevalent with a common cold than with influenza. These infections can be analyzed based on the fact that signatures of sinusitis, such as fluid in the sinuses, can increase the indication precision to distinguish between respiratory infection types. Furthermore, common cold viruses usually do not cause substantial damage to the airway epithelium, whereas influenza and COVID-19 can damage cells in the respiratory epithelium. In fact, a broad variety of respiratory pathogens, including rhinoviruses, coronaviruses, and the like, can adversely affect cells. Redness and inflammation associated with such cellular damage can be detected using the disclosed techniques. The disclosed techniques can be applied when looking into a patient's throat and taking images to measure fluorescence, absorption, and thermal radiation. As a result, possible infection from respiratory viruses such as SARS-CoV-2, Influenza A, and Influenza B can be detected. Such methods can also facilitate telemedicine diagnostics. In embodiments, the indication enables infection detection. In embodiments, the infection detection is based on biochrome identification. In embodiments, the indication enables respiratory infection detection. In embodiments, the respiratory infection detection comprises influenza detection. In embodiments, the influenza detection comprises COVID-19 detection.

This technology isolates signals from infection-associated biochromes, such as Porphyrin and Pyoverdine, by holding an excitation wavelength constant and collecting signals from progressively longer wavelength emission channels. This action is performed at each pixel in an image. In one embodiment, fluorescence is collected by exciting wavelengths in the blue/UV region of the spectrum such that the peak of the spectral distribution of the excitation source is at a lower wavelength (higher energy) than what is typically detected by the sensor (CMOS or CCD as examples) that is being used for detecting photons and generating an image.

The indication can enable residual cancer detection 438. Autofluorescence imaging is enabled by the disclosed concepts and has been used to diagnose oral cancer, breast cancer, lung cancer, skin cancer, brain cancer, and others. Autofluorescence from NADH has been cited as one possible biomarker for targeting cancer. Similarly, fluorescence from dense connective tissue (extracellular matrix, etc.) associated with a tumor can be used to delineate tumor boundaries. In addition, such techniques can enable detection of residual cancer during surgery. In embodiments, the indication enables residual cancer detection. In further embodiments, the residual cancer detection occurs during oncological surgery.

The indication can further enable food recognition, food quality, or food safety 440. Common foodborne pathogens include E. coli, Salmonella, Listeria, Cyclospora, and Hepatitis A. Disclosed techniques can enable rapid detection of foodborne pathogens in order to avoid distribution of contaminated foods. Authentication, quality, and possible adulteration of food must be monitored for distribution and consumption. For example, liquor, wine, and beer inspection can be performed by analyzing both water content and the presence of fluorescent compounds. Fluorescent compounds such as polyphenols, flavonoids, stilbenes, tannins, coumarins, and fluorescent amino acids are key markers of authenticity and quality. In some embodiments, two or three excitation LEDs at different blue and UV wavelengths may be employed for determining a shift in emission resulting from a change in excitation frequency. Such techniques can be used in plant food quality analysis, milk quality analysis, fruit quality analysis, coffee quality analysis, as well a protein quality analysis of products as varied as beef and sashimi, to name just a few. Other applications include monitoring the progress of fermentation, such as malolactic fermentation, for the deacidification of red wines. In-line monitoring of the fermentation process can also be applied to fermentation processes in which yeast or bacteria are programmed to produce a specific chemical such as THC and CBD. In addition, monitoring caloric intake can be enabled by food composition and rough, overall portion size identification. In embodiments, the indication of composition of the material sample includes identification of the presence of water, and the presence of water is used to determine organism health for the material sample. In embodiments, the indication enables food recognition, food quality, or food safety identification. In embodiments, the food quality detects food adulteration. And in embodiments, the food quality monitors progression of fermentation.

The indication can enable agricultural yield optimization 442. Especially in automated indoor farming, which is poised to assume a significant burden of the food supply, the disclosed techniques can enable identification of crop ripeness, crop water sufficiency, crop fertilization sufficiency, crop disease detection, and so on. This approach can enable minimized use of insecticides and herbicides while optimizing crop yield. In addition, a robot- or drone-based approach to agricultural optimization is feasible due to the portable attributes of the disclosed techniques. In embodiments, the indication enables agricultural yield optimization. In further embodiments, providing excitation and measuring RGB sensor output values are accomplished using drone technology. The indication can have applications in law enforcement and can enable a field sobriety evaluation 444 for an individual. A contactless evaluation using the disclosed techniques can determine the need for a more invasive breathalyzer test. In addition to visual indicators such as enlarged pupils and eye movement that is faster than normal, measured amounts of vasoconstriction and vasodilation, depending on a level of intoxication, can be enabled using the indication. In embodiments, the indication enables field sobriety evaluation of individuals. In embodiments, the field sobriety evaluation of individuals is accomplished in a contactless manner. The indication can have further applications in dental care. The indication can enable an oral hygiene evaluation 446 for an individual. This can include detecting plaque, gingivitis, and other dental abnormalities using multispectral imaging and fluorescence. Thus, in embodiments, the indication enables oral hygiene evaluation.

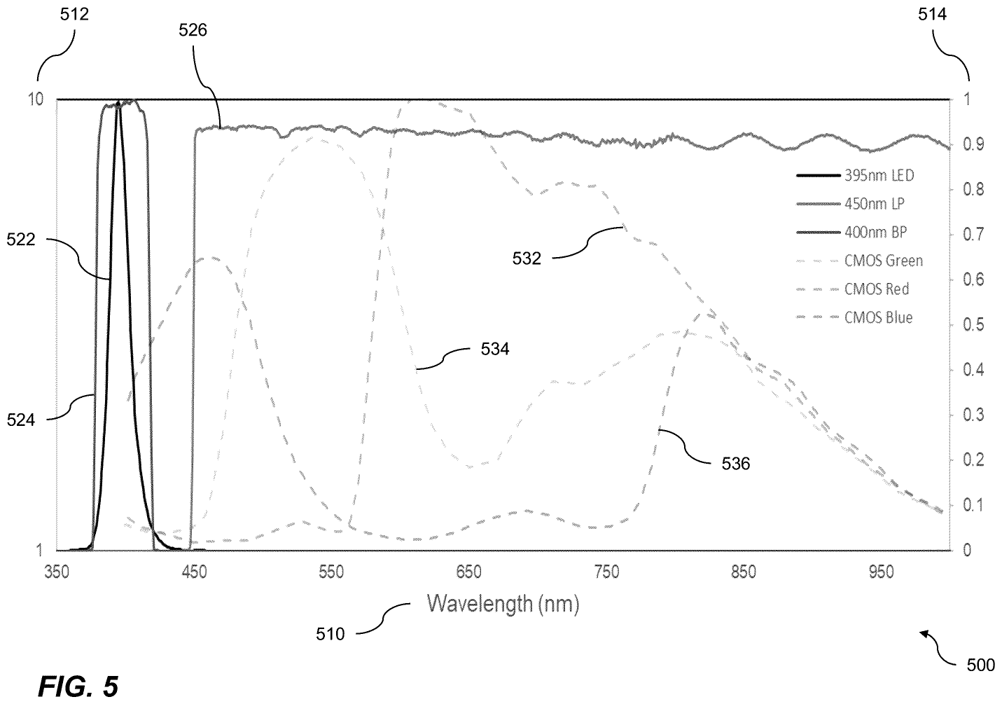

FIG. 5 is a graph showing fluorescence measurements. Measurements of fluorescence responses of materials illuminated by various light spectrum wavelengths can be useful to understanding the composition of a material sample. The material sample can be associated with a skin wound such as skin wound exudate. The fluorescence measurements enable wound care image analysis using a skin wound factor. Various material samples, such as living organism samples, tissue samples, blood samples, skin samples, wound samples, infection samples, food samples, dental or oral hygiene samples, inanimate object samples, and so on can have fluorescence measurement techniques performed on them. To accomplish fluorescence measurements, images of fluorescent responses of materials to various light spectra can be used. The images can contain fluorescence characteristics and reflectance characteristics associated with the material sample. One or more skin wound images for an individual are obtained, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors.

In the graph 500, an x-axis indicating wavelength 510 is provided. Increasing wavelength from left to right indicates decreasing frequency of light waves and a traversal from the ultraviolet spectrum, roughly sub-400 nm, through the blue, green, and red wavelength regions, roughly 450 nm, 550 nm, and 650 nm, respectively, to the infrared wavelength band, which is roughly greater than 750 nm. It should be noted that an exact wavelength definition of a particular color is somewhat arbitrary and dependent on the sensor type. For example, the cones of a human eye roughly sense RGB signals using three cone types, but they are generally distributed differently from a typical CMOS RGB sensor's output. However, maintaining a consistent definition for a given system is generally required in order to provide consistent sample indications. The graph 500 also includes a left y-axis of absorption amount 512 from one to ten and a right y-axis of transmission amount 514 from zero to one.

The graph 500 includes an excitation wavelength 522. The excitation wavelength 522 is centered substantially at 405 nm in the ultraviolet light wavelength spectrum. Note that wavelength 522 is a relatively narrow excitation, but that due to practical considerations, energy tails of the excitation wavelength can sometimes extend up toward the visible light RGB spectrum at 450 nm and above. To prevent bleed-over into the RGB spectrum, a bandpass filter, indicated by transmission spectrum 524, can be included. The bandpass filter can help attenuate excitation wavelengths outside of the band, such as a 50 nm bandpass filter centered at 400 nm. To further prevent bleed-over into the RGB spectrum, a long-pass filter, indicated by absorption spectrum 526, can be included. It should be noted that the bandpass optical filters can be placed between an excitation source and a sample, and the long-pass optical filter can be placed between the sample and an RGB sensor. In this manner, the excitation wavelength does not “bleed over” and affect the fluorescence measurements of wavelengths being emitted by the illuminated sample.

The graph 500 includes RGB sensor characteristics, such as sensor characteristic 532, indicative of the “R” or red output of an RGB sensor, sensor characteristic 534, indicative of the “G” or green output of an RGB sensor, and sensor characteristic 536, indicative of the “B” or blue output of an RGB sensor. The RGB outputs represented by characteristics 532, 534, and 536 can be used directly or can be compensated (as discussed elsewhere) to enable generation of an indication of material composition.

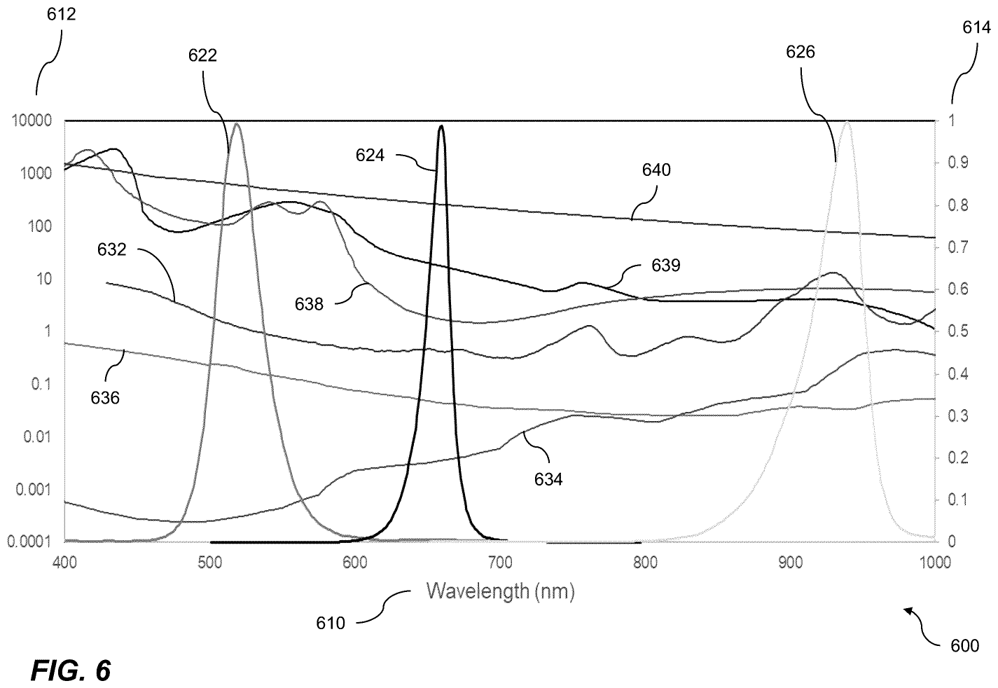

FIG. 6 is a graph illustrating biochrome absorption. Absorption characteristics such as biochrome absorption characteristics can be useful to understanding the identification of biochromes and water in a living organism sample. Biochrome absorption and water absorption enable wound care image analysis using skin wound factors. In the graph 600, an x-axis indicating wavelength 610 is provided. Increasing wavelength from left to right indicates decreasing frequency of light waves and a traversal from the ultraviolet spectrum, roughly sub-400 nm, through the blue, green, and red wavelength regions, roughly 450 nm, 550 nm, and 650 nm, respectively, to the infrared band, which is roughly greater than 750 nm. It should be noted that an exact wavelength definition of a particular color is somewhat arbitrary and dependent on the sensor type. For example, the cones of a human eye roughly sense RGB signals using three cone types, but they are generally distributed differently from a typical CMOS RGB sensor's output. However, maintaining a consistent definition for a given system is generally required in order to provide consistent sample indications. The graph 600 also includes a logarithmic left y-axis of absorption 612 and a linear right y-axis of normalized excitation 614.

The graph 600 illustrates three excitation wavelengths for sample illumination. Excitation wavelength 622 is substantially centered at a wavelength of about 523 nm; excitation wavelength 624 is substantially centered at a wavelength of about 660 nm; and excitation wavelength 626 is substantially centered at a wavelength of about 940 nm. Thus, the three excitation wavelengths, wavelength 622, wavelength 624, and wavelength 626, are spaced at least 100 nm apart over an extended visible light spectrum. The sharp, bell curve shape of the excitations provides for little to no overlap of those excitation wavelengths. Also, it can be noted that 940 nm light is sometimes considered to be near-infrared (NIR) wavelength light. However, most silicon-based CMOS sensors detect 940 nm light. In some usage scenarios, a short-pass filter is applied to prevent noise from NIR photons if that wavelength is not being used by an application. Nonetheless, a 940 nm wavelength can be considered part of an extended visible light spectrum and included when discussing RGB sensor usage.

The graph 600 includes various biochrome and water absorption characteristics, such as a hemoglobin (Hgb or deoxyHb) absorption characteristic 632 and a water absorption characteristic 634. By taking the value of each absorption characteristic line on graph 600 at each of the three excitation wavelengths 622, 624, and 626, a tri-valued metric can be determined. Notably, while many of the biochrome absorption characteristics wander about with no simple trend across increasing wavelength, such as is observed for Hgb absorption characteristic 632, the water absorption characteristic 634 displays a monotonically increasing metric across increasing wavelength excitations 622, 624, and 626, with metric increases close to three orders of magnitude across the excitations.

The graph 600 shows other absorption characteristics, such as melanin absorption characteristic 640, fat absorption characteristic 639, oxygenated hemoglobin (oxyHb) absorption characteristic 638, and collagen absorption characteristic 636. The graph 600 thus illustrates a method for multispectral sample analysis comprising: providing at least two excitation light wavelengths to a material sample, wherein the material sample exhibits absorption characteristics along the Red-Green-Blue (RGB) light wavelength spectrum; measuring output values of an RGB sensor, wherein the measuring detects the absorption characteristics of the material sample, and wherein the absorption characteristics are in response to the at least two excitation light wavelengths; and generating an indication of composition of the material sample, wherein the indication is based on interpreting the output values that were measured.

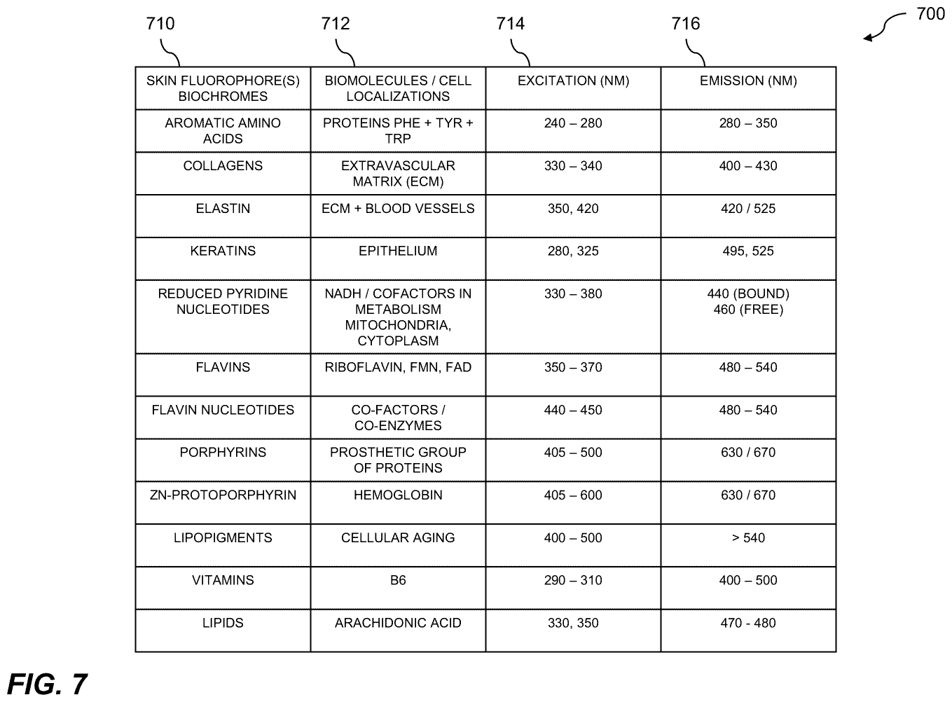

FIG. 7 is a table showing biochromes and fluorescent channels. A fluorophore is a chemical compound that when excited by light, re-emits light. Biochromes include substances such as chemical compounds which present a color based on selective absorption of other colors. The colors that are presented and selectively absorbed are based on wavelengths of light. Biochromes and fluorescent channels support wound care image analysis using skin wound factors. One or more skin wound images for an individual are obtained, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors.

A table based on biochromes and fluorescent channels is shown 700. The table includes skin fluorophores and biochromes 710. Each of the skin fluorophores and biochromes can correspond to a factor associated with healing. Each of the factors can further be associated with one or more biomolecules or cell localizations 712. The various skin fluorophores and biochromes can be excited by an optical excitation light wavelength band 714. The excited fluorophores and biochromes generate an emission response 716 by the scanned material sample. In embodiments, the material sample can include cells, tissues, and organs. The material sample can include skin, lungs, and so on. The emission response can be indicative of infection such as infection of a wound, respiratory infection such as a COVID-19 infection or influenza infection, residual cancer detection, and so on.

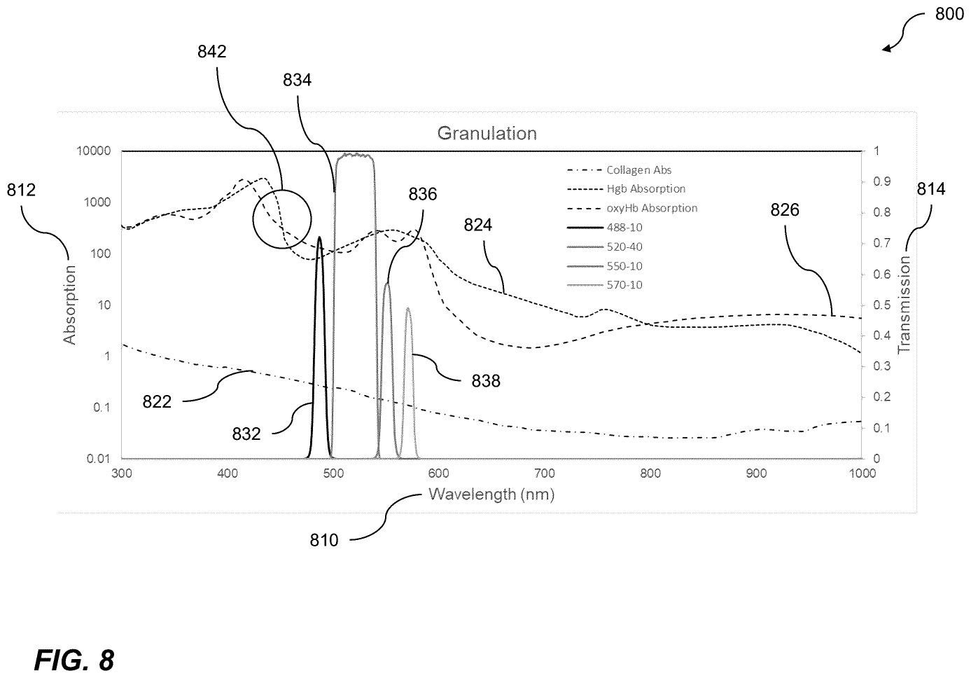

FIG. 8 shows granulation identification. Using disclosed techniques, the graph 800 shows an output status for identifying granulation in tissue. An output indicative of a biophysical skin wound status is enabled by image analysis using skin wound factors. The biophysical skin wound status can include a wound topology based on a measure of granulation. One or more skin wound images for an individual are obtained, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors.

In the graph 800, an x-axis indicating wavelength 810 is provided. Increasing wavelength from left to right indicates decreasing frequency of light waves and a traversal from the ultraviolet spectrum, approximately sub-400 nm, through the blue, green, and red wavelength regions, roughly 450 nm, 550 nm, and 650 nm, respectively, to the infrared wavelength band, which is roughly greater than 750 nm. It should be noted that an exact wavelength definition of a particular color is somewhat arbitrary and is dependent on the sensor type. For example, the cones of a human eye roughly sense RGB signals using three cone types, but they are generally distributed differently from a typical CMOS RGB sensor's output. However, maintaining a consistent definition for a given system is generally required in order to provide consistent sample indications. The graph 800 also includes a left y-axis of absorption amount 812 and a right y-axis of transmission amount 814.

The graph 800 includes absorption characteristics, such as absorption characteristic 822, typical for the presence of collagen, absorption characteristic 824, typical for the presence of hemoglobin (Hgb or deoxyHb), and absorption characteristic 826, typical for the presence of oxygenated hemoglobin (oxyHb). The typical absorption characteristics 822, 824, and 826 can be used as reference signals as is, or they can be compensated to enable generation of an indication of material composition. A granulation output signature is isolated by quantifying the dip in the Hgb spectrum at 450 nm, illustrated in highlight circle 842. Tissue that is mostly collagen will not have a dip, whereas granulation tissue will have a dip that resides roughly halfway between the typical Hgb characteristic 824 and/or 826 and the typical collagen characteristic 822. The intensity of the dip can be quantified by taking the ratio or difference of the absorption intensity at 836 and/or 838 and comparing it to the absorption intensity at 834 and/or 832.

The graph 800 shows signals indicative of filter characteristics, including signals 832, 834, 836, and 838. Signal 832 represents a light wavelength centered at about 488 nm with a bandwidth of about 10 nm and a relative transmission amplitude of about 0.7 out of 1. Signal 834 represents a light wavelength centered at about 520 nm with a bandwidth of about 40 nm and a relative transmission amplitude of almost 1.0 out of 1. Signal 836 represents a light wavelength centered at about 550 nm with a bandwidth of about 10 nm and a relative transmission amplitude of about 0.6 out of 1. Signal 838 represents a light wavelength centered at about 570 nm with a bandwidth of about 10 nm and a relative transmission amplitude of about 0.5 out of 1. It is to be understood that the characteristics and signals illustrated herein have substantially the values shown in graph 800, but that normal, typical variations and equipment calibration may provide a delta, such as a five percent delta, to the characteristics and signals.

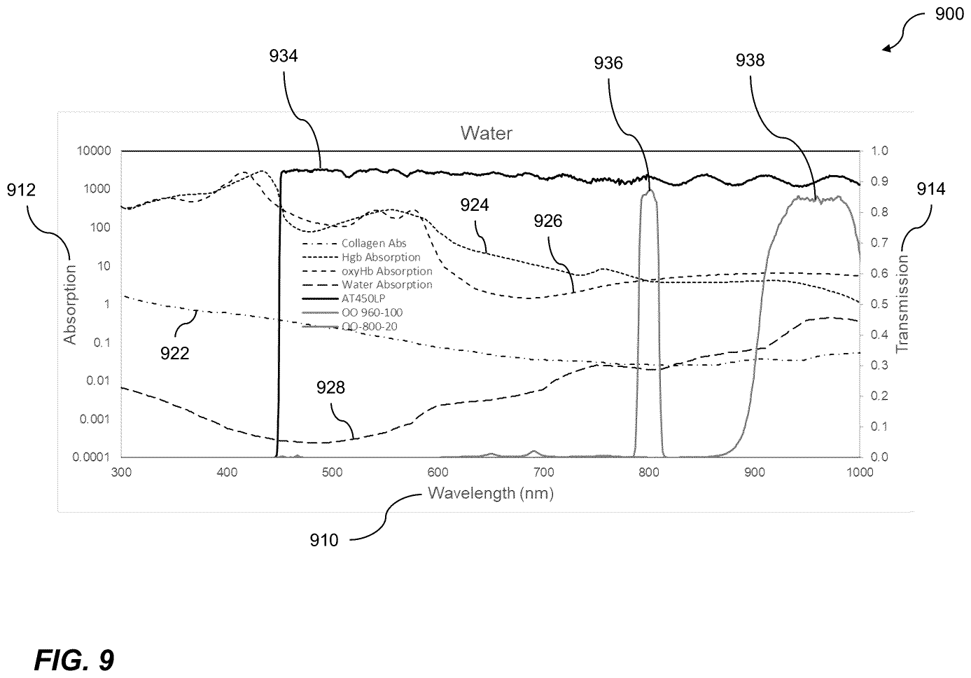

FIG. 9 shows water identification. Using disclosed techniques, the graph 900 shows a generated output for identifying the presence of water in tissue. A generated output, which can be indicative of biophysical skin wound status, is enabled by wound care image analysis using skin wound factors. One or more skin wound images are obtained for an individual, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors.

In the graph 900, an x-axis indicating wavelength 910 is provided. Increasing wavelength from left to right indicates decreasing frequency of light waves and a traversal from the ultraviolet spectrum, roughly sub-400 nm, through the blue, green, and red wavelength regions, roughly 450 nm, 550 nm, and 650 nm, respectively, to the infrared wavelength band, which is roughly greater than 750 nm. It should be noted that an exact wavelength definition of a particular color is somewhat arbitrary and dependent on the sensor type. For example, the cones of a human eye roughly sense RGB signals using three cone types, but they are generally distributed differently from a typical CMOS RGB sensor's output. However, maintaining a consistent definition for a given system is generally required in order to provide consistent sample indications. The graph 900 also includes a left y-axis of absorption amount 912 and a right y-axis of transmission amount 914.

The graph 900 includes absorption characteristics, such as absorption characteristic 922, indicative of the presence of collagen, absorption characteristic 924, indicative of the presence of hemoglobin (Hgb or deoxyHb), absorption characteristic 926, indicative of the presence of oxygenated hemoglobin (oxyHb), and absorption characteristic 928, indicative of the presence of water. The typical absorption characteristics 922, 924, 926, and 928 can be used as reference signals as is, or they can be compensated to enable generation of an indication of material composition. A water output signature is isolated by irradiating a material sample (wound or other) with broad-band white light and comparing the absorption at 960 nm (or similar) and 800 nm (or similar). In the graph 900, broad-band white light is passed by a long-pass filter, shown as signal 934, which cuts off wavelengths of light below 450 nm (in the ultraviolet range), and allows visible light above 450 nm with a relative transmission amplitude of about 0.9 out of 1. Light emanating from the sample can be measured at two or more points at successively longer wavelengths, as shown by signals 936 and 938. Signal 936 represents an optical filter centered at about 800 nm with a bandwidth of about 20 nm and a relative amplitude of about 0.85 out of 1. Signal 938 represents a light wavelength centered at about 960 nm with a bandwidth of about 100 nm and a relative amplitude of about 0.85 out of 1. The output signature of water will be substantially higher at the longer wavelength sample point. As shown in graph 900, this is not true for the absorption characteristics of collagen 922 and hemoglobin 924 and 926 as sampled at 800 nm and 960 nm. It is to be understood that the characteristics and signals have substantially the values illustrated in graph 900, but that normal, typical variations and equipment calibration may provide a delta, such as a five percent delta, to the characteristics and signals.

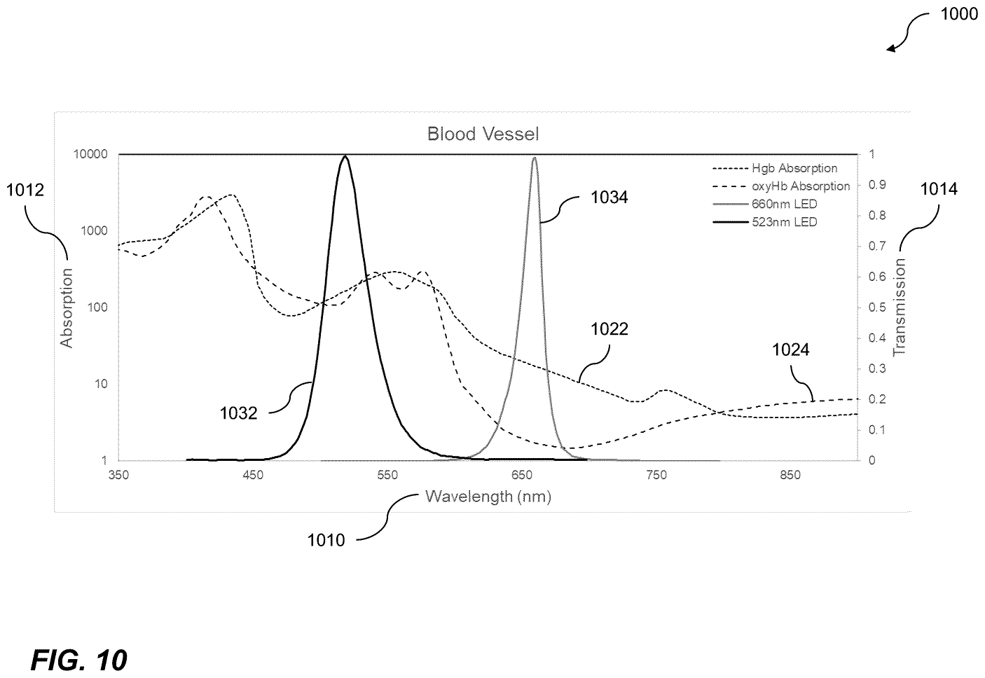

FIG. 10 shows blood vessel identification. Using disclosed techniques, the graph 1000 shows an output signature that can be used for identifying the presence of blood vessels in tissue. An output, such as an output indicative of biophysical skin wound status, can be generated. The output wound status generation is based on analysis of skin wound images to determine metrics for skin wound factors. Output biophysical wound status usage is enabled by image analysis using wound factors. One or more skin wound images are obtained for an individual, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors.

In the graph 1000, an x-axis indicating wavelength 1010 is provided. Increasing wavelength from left to right indicates decreasing frequency of light waves and a traversal from the ultraviolet spectrum, roughly sub-400 nm, through the blue, green, and red wavelength regions, roughly 450 nm, 550 nm, and 650 nm, respectively, to the infrared wavelength band, which is roughly greater than 750 nm. It should be noted that an exact wavelength definition of a particular color is somewhat arbitrary and dependent on the sensor type. For example, the cones of a human eye roughly sense RGB signals using three cone types, but they are generally distributed differently from a typical CMOS RGB sensor's output. However, maintaining a consistent definition for a given system is generally required in order to provide consistent sample indications. The graph 1000 also includes a left y-axis of absorption amount 1012 and a right y-axis of transmission amount 1014.

The graph 1000 includes absorption characteristics, such as absorption characteristic 1022, indicative of the presence of hemoglobin (Hgb or deoxyHb), and absorption characteristic 1024, indicative of the presence of oxygenated hemoglobin (oxyHb). The typical absorption characteristics 1022 and 1024 can be used as reference signals as is, or they can be compensated to enable generation of an indication of material composition. A blood vessel signal is isolated based on the absorption of Hgb at 523 nm and 660 nm. Light from two different light emitting diodes (LEDs) is used to illumine a material sample. In the graph 1000, signal 1032 represents the typical output spectrum of a 523 nm LED, and signal 1034 represents the typical output spectrum of a 660 nm LED. As can be seen in graph 1000, the absorption characteristics of Hgb 1022 and oxyHb 1024 are very similar at about 523 nm. However, an order of magnitude difference is observed when comparing absorption characteristics 1022 and 1024 at 660 nm. The low absorption in the red band is indicative of oxygenated hemoglobin, which is typically found in active blood vessels, but not found in pooled blood due to bruising or other wound-related phenomena. It is to be understood that the characteristics and signals have substantially the values illustrated in graph 1000, but that normal, typical variations and equipment calibration may provide a delta, such as a five percent delta, to the characteristics and signals.

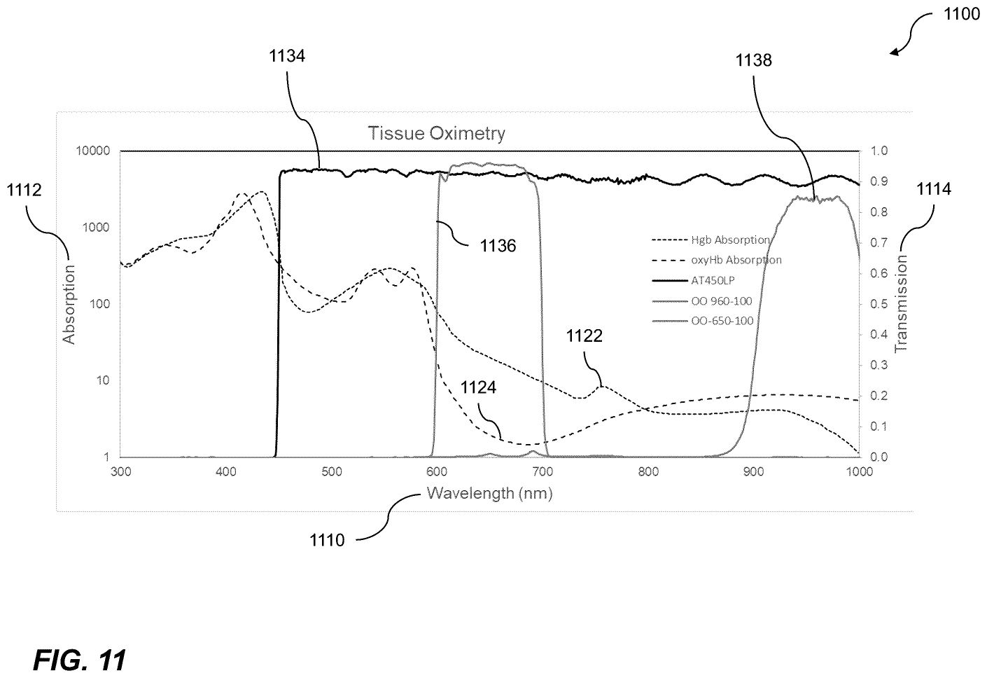

FIG. 11 shows tissue oximetry. Using disclosed techniques, the graph 1100 shows an output signature for identifying the presence of oxygen in tissue. An output can be presented on a display as a graphical rendering. The output that is indicative of biophysical skin wound status can be generated. The output wound status generation is based on analysis of skin wound images to determine metrics for skin wound factors. Output biophysical status usage is enabled by image analysis using wound factors. One or more skin wound images for an individual are obtained, wherein the skin wound images provide light spectrum information that includes fluorescence characteristics and reflectance characteristics. One or more processors are used to analyze the skin wound images to determine metrics for skin wound factors, based on the light spectrum information, wherein the skin wound factors comprise infection, inflammation, granulation, topology, and epithelialization. An output indicative of biophysical skin wound status is generated, wherein the output is based on the analyzing of the skin wound factors.

In the graph 1100, an x-axis indicating wavelength 1110 is provided. Increasing wavelength from left to right indicates decreasing frequency of light waves and a traversal from the ultraviolet spectrum, roughly sub-400 nm, through the blue, green, and red wavelength regions, roughly 450 nm, 550 nm, and 650 nm, respectively, to the infrared wavelength band, which is roughly greater than 750 nm. It should be noted that an exact wavelength definition of a particular color is somewhat arbitrary and dependent on the sensor type. For example, the cones of a human eye roughly sense RGB signals using three cone types, but they are generally distributed differently from a typical CMOS RGB sensor's output. However, maintaining a consistent definition for a given system is generally required in order to provide consistent sample indications. The graph 1100 also includes a left y-axis of absorption amount 1112 and a right y-axis of transmission amount 1114.

The graph 1100 includes absorption characteristics, such as absorption characteristic 1122, indicative of the presence of hemoglobin (Hgb or deoxyHb), and absorption characteristic 1124, indicative of the presence of oxygenated hemoglobin (oxyHb). The typical absorption characteristics 1122 and 1124 can be used as reference signals as is, or they can be compensated to enable generation of an indication of material composition. An oxygenated tissue output signature is isolated by comparing absorption at 960 nm, where oxyHb absorption dominates, to 650 nm, where Hgb absorption dominates. In the graph 1100, broad-band white light is passed by a long-pass filter, shown as signal 1134, which cuts off wavelengths of light below 450 nm (in the ultraviolet range), and allows visible light above 450 nm with a relative transmission amplitude of about 0.9 out of 1. Light emanating from the sample can be measured at two or more points at successively longer wavelengths, as shown by signals 1136 and 1138. Signal 1136 represents an optical filter centered at about 650 nm with a bandwidth of about 100 nm and a relative amplitude of about 0.95 out of 1. Signal 1138 represents a light wavelength centered at about 960 nm with a bandwidth of about 100 nm and a relative amplitude of about 0.85 out of 1. The output signature of oxygenated tissue will show higher absorption at the longer wavelength sample point. It is to be understood that the characteristics and signals have substantially the values illustrated in graph 1100, but that normal, typical variations and equipment calibration may provide a delta, such as a five percent delta, to the characteristics and signals.

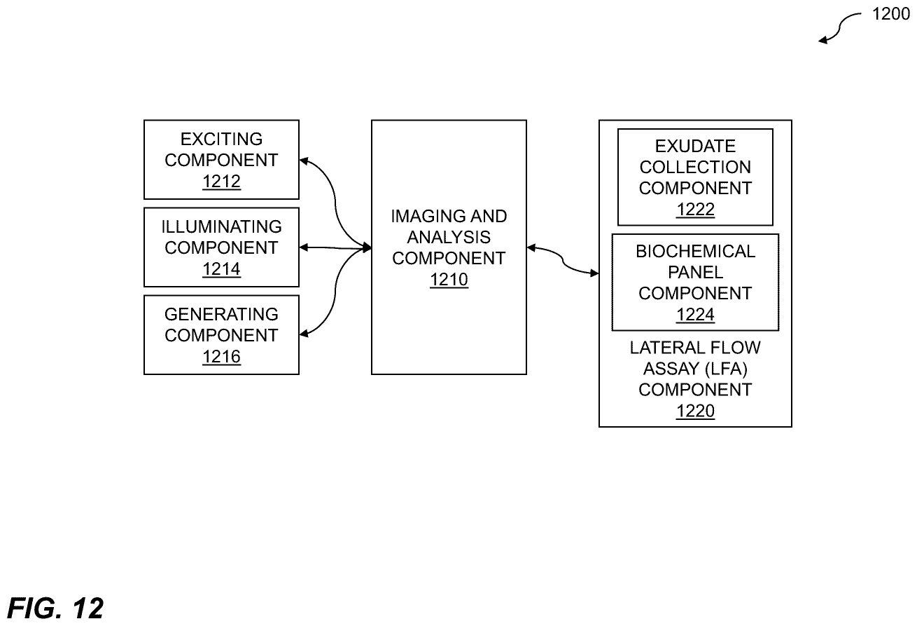

FIG. 12 is a system block diagram for a lateral flow assay. A lateral flow assay is based on the use of a substrate such as a nitrocellulose membrane. The lateral flow assay can enable a biochemical assay. The biochemical assay can be performed on wound analytes. The lateral flow assay can be used to confirm the presence or absence of a target analyte such as a pathogen, biomarker, and so on within mixtures. The mixtures can include complex mixtures. The complex mixtures can include wound exudates. A lateral flow assay can support wound care by identifying analytes, pathogens, biomarkers, etc. within a mixture. The wound care can be further enabled by image analysis using skin wound factors. A lateral flow assay can be analyzed in conjunction with an output associated with a material sample comprising a skin wound. The output can include a generated output that is indicative of biophysical skin wound status. The generated output is based on analysis of skin wound images obtained for an individual. The skin wound images provide light spectrum information that includes fluorescence characteristics and reflectance characteristics. The analysis determines metrics for skin wound factors, where the skin wound factors comprise infection, inflammation, granulation, topology, and epithelialization.

The system block diagram 1200 includes an imaging and analysis component 1210. The imaging and analysis component can provide one or more light bandwidth sources that can be used to illuminate a material sample. The material sample can include a skin wound, the exudate of the skin wound, and so on. The one or more light bandwidth sources can include far-infrared, mid-infrared, and near-infrared sources; visible light sources such as red, green, and blue sources; and so on. The analysis can include analysis of one or more responses of the material sample to the light-bandwidth sources. The response can include fluorescence, reflectance, absorption, and so on (discussed below). The system block diagram 1200 can further include an exciting component 1212. The exciting component 1212 can excite a light wavelength on a material sample. The material sample can include a skin wound, wound exudate, and so on. The exciting enables capture of fluorescence characteristics of the material sample. The fluorescence characteristics can include light wavelengths within one or more infrared light wavelengths, visible light wavelengths, and so on. The material sample can exhibit fluorescence characteristics along the Red-Green-Blue (RGB) light wavelength spectrum. The fluorescence characteristics can be captured using a sensor such as an RGB sensor. In embodiments, the light wavelength that excites fluorescence characteristics of the material sample can be substantially a 395 nm light wavelength.

The system block diagram 1200 can include an illuminating component 1214. The illuminating component 1214 can illuminate the material sample with at least three additional light wavelengths. The additional light wavelengths can include infrared light wavelengths, visible light wavelengths, etc. In embodiments, the at least three additional light wavelengths can include a blue-band light wavelength, a green-band light wavelength, and a red-band light wavelength. The blue-, green-, and red-band light can comprise various light wavelengths. In embodiments, the blue-band light wavelength can be substantially a 460 nm wavelength; the green-band light wavelength can be substantially a 523 nm wavelength; and the red-band light wavelength can be substantially a 660 nm wavelength. Other light wavelengths can be used. Further embodiments can include illuminating the material sample with at least one further additional light wavelength. The at least one further additional light wavelength can include infrared light, visible light, etc. In embodiments, the at least one further additional light wavelength can include an infrared-band light wavelength. Various infrared-band light wavelengths can be used. In embodiments, the infrared-band light wavelength can be substantially a 940 nm wavelength. The illuminating enables capture of reflectance characteristics of the material sample. The reflectance characteristics can include infrared light, visible light, and the like. The material sample can exhibit reflectance characteristics along the Red-Green-Blue (RGB) light wavelength spectrum. The reflectance characteristics can be captured using a sensor such as an RGB sensor.

The system block diagram 1200 can include a generating component 1216. The generating component 1216 can generate an output indicative of biophysical status of the material sample. The output that is generated is based on analysis of the fluorescence characteristics and the reflectance characteristics. The analysis can be accomplished using the imaging and analysis component 1210. The output that is generated can include one or more numerical values, text, and so on. The output can be rendered on a display such as a display associated with a computer, tablet computer, smartphone, or other display associated with an electronic device. The imaging and analysis component 1210 can include a thermal imaging component. The thermal imaging component can be used to capture a thermal image of the material sample. The captured thermal image can include a thermal image of a skin wound. The thermal image can be analyzed using the imaging and analysis component. Further embodiments include augmenting the output that was generated. The augmenting can be based on an analysis of the thermal image.

The system block diagram 1200 can include a lateral flow assay (LFA) component 1220. Discussed above and throughout, a lateral flow assay is a technique that can be used to detect the presence or the absence of a target analyte. It can also quantify the amount or concentration of a target analyte or indicate the presence above or below a particular designed threshold. An analyte can include a substance such as a chemical substance that can be detected, identified, measured, and so on. The LFA can include a control line, where the control line can be used to determine whether the LFA is working or not. One or more additional lines can appear to indicate the presence of one or more analytes within a test sample. LFAs can be commonly used to gauge patient health by detecting disease such as COVID-19. LFAs are also commonly used for in-home tests such as pregnancy tests. The lateral flow assay component can include or be coupled to other components. The system block diagram 1200 can include an exudate collection component 1222. The exudate collection component can be used to collect drainage or exudate from a skin wound. Exudate can further be collected from a bandage, gauze pad, a negative pressure wound fluid collection device, and so on that was covering a skin wound. The system block diagram 1200 can include a biochemical panel component 1224. The biochemical panel component can include a membrane such as a nitrocellulose membrane, labels such as colored nanoparticles, antibodies, and so on. The biochemical panel component may further include an applicator to apply a substance to the LFA, a reader to detect test results, and so on.

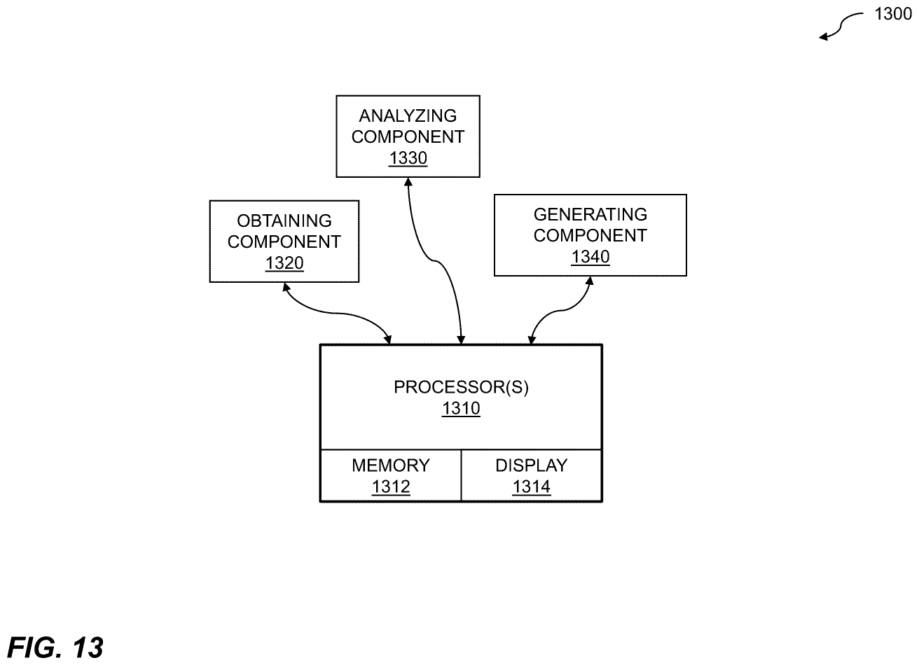

FIG. 13 is a system diagram for image analysis using skin wound factors. The system 1300 can include one or more processors 1310, which are coupled to a memory 1312 which stores instructions. The system 1300 can further include a display 1314 coupled to the one or more processors 1310 for displaying data, indications of sample analysis, skin wound factors, directions, input requests, control options, excitation wavelengths, filter options, compensation options, data forwarding options, and so on. Embodiments of the system 1300 comprise a computer system for image analysis comprising: a memory 1312 which stores instructions; one or more processors 1310 that are coupled to the memory, wherein the one or more processors, when executing the instructions which are stored, are configured to: obtain one or more skin wound images for an individual, wherein the skin wound images provide light spectrum information that includes fluorescence characteristics and reflectance characteristics; analyze, using one or more processors, the skin wound images to determine metrics for skin wound factors, based on the light spectrum information, wherein the skin wound factors comprise infection, inflammation, granulation, topology, and epithelialization; and generate an output indicative of biophysical skin wound status, wherein the output is based on the analyzing of the skin wound factors.

The system 1300 can include an obtaining component 1320. The obtaining component 1320 can be used to obtain one or more skin wound images for an individual. The skin wound images provide light spectrum information that includes fluorescence characteristics and reflectance characteristics. Other light spectrum information can be obtained for the individual. In embodiments, the light spectrum information can include infrared thermal characteristics. The skin wound can be illuminated by one or more wavelengths of light in order to obtain the images. The one or more light wavelengths can include light wavelengths generated by various sources including an incandescent light source, an LED light source, a Red-Green-Blue (RGB) light source, a laser light source, and so on. The light source or sources can emit a narrow spectrum of light at primarily one wavelength, at primarily two or more wavelengths, across a broad spectrum of multiple wavelengths, in the visible spectrum, in the infrared spectrum, in the ultraviolet spectrum, and so on. The light source wavelength or wavelengths can enable material sample fluorescence, reflectance, absorption, and so on. The light spectrum information enables obtaining fluorescence characteristics and reflectance characteristics from the skin wound images. In embodiments, the light spectrum information can include light along the Red-Green-Blue (RGB) light wavelength spectrum. In embodiments, light spectrum information can include wavelengths of 523 nm, 660 nm, and 940 nm. Other wavelengths can also be included such as wavelengths associated with fluorescence characteristics. In embodiments, the other light wavelength associated with fluorescence characteristics can be substantially a 395 nm light wavelength. Further embodiments include obtaining additional skin wound images for the individual. The additional skin wound images can include substantially similar or substantially dissimilar light spectrum information. The additional skin wound images can be captured over a period of time. In embodiments, the additional skin wound images can provide a time-sequenced representation of skin wound healing.

The system 1300 can include an analyzing component 1330. The analyzing component 1330 can analyze, using one or more processors, the skin wound images to determine metrics for skin wound factors, based on the light spectrum information, wherein the skin wound factors comprise infection, inflammation, granulation, topology, and epithelialization. The one or more processors that can be used for the analyzing can include a handheld device such as a smartphone or tablet, a specialized handheld device intended for skin wound image analysis, a desktop computer, a laptop computer, a server, a cloud-based server, etc. The wound factors can be analyzed singly, collectively, and so on. In embodiments, the analyzing can be based on a subset of the skin wound factors. The skin wound factors can be interdependent. In embodiments, an epithelialization metric can be included in determining skin wound topology. A skin wound factor can include one or more measurements, assessments, and the like. In embodiments, the skin wound topology can include wound area, wound volume, wound depth, wound shape, wound edge definition, wound texture, or wound topography. In other embodiments, the wound topology can be identified based on a measure of granulation, slough, eschar, flavin adenine dinucleotide (FAD), and/or nicotinamide adenine dinucleotide plus hydrogen (NADH) in the wound sample. The metrics that are analyzed can be based on one or more signatures. In embodiments, the metrics can include physical and chemical skin wound signatures. The analyzing can be based on samples, where the samples can be collected from one or more bandages associated with an individual. Bandages associated with more than individual can be used to obtain samples. Further embodiments include obtaining additional bandage samples corresponding to skin wounds on a plurality of individuals.

In other embodiments, the analysis can include identifying epithelialization within the material sample. Epithelialization can include a migration of epithelial cells upwards to repair a damaged or wounded area of the body such as a skin wound. The identifying epithelization can indicate a level of healing of a skin wound. In embodiments, the epithelialization can be identified based on a measure of nicotinamide adenine dinucleotide (NAD), NADH, and/or FAD. In other embodiments, the analysis can include identifying granulation within the material sample. The identifying granulation can include identifying an amount of granulation tissue, where the granulation tissue can fill in a wound. In embodiments, the granulation can be identified based on a measure of hemoglobin and collagen. In further embodiments, the analysis can include identifying infection within the material sample. Identifying infection is critical to wound treatment. In embodiments, the infection can be identified based on a measure of porphyrin, pyoverdine, slough, eschar, or inflammation signature. The inflammation signature can be based on temperature, liquid content, and so on. In embodiments, the inflammation signature can include wound temperature, wound water content, and periwound redness. The various analyses can contribute to an N-factor biophysical status of a material sample. In embodiments, identifying wound topology, inflammation, epithelialization, granulation, and infection can comprise a five-factor biophysical material sample status.

The system 1300 can include a generating component 1340. The generating component 1340 can generate an output indicative of biophysical skin wound status, wherein the output is based on the analyzing of the skin wound factors. The generating component can provide the output based on analysis of the fluorescence characteristics and the reflectance characteristics of the skin wound factors. The output indicative of biophysical skin wound status can be rendered a display, where the display can be associated with a handheld device, a purpose-built handheld device, a desktop computer, a laptop computer, and so on. The output that is generated can be used for a variety of purposes such as gauging the extent or severity of a wound, measuring wound healing, and the like. Further embodiments include developing a skin wound care plan, based on the output. The skin wound care plan can include a cleansing protocol or recommendation, a type of wound dressing and frequency of dressing change, etc. Discussed previously, additional skin wound images for the individual can be obtained and analyzed. Further embodiments include generating a wound healing trajectory, based on the one or more skin wound images and at least one additional skin wound image. Wound healing can be thought to comprise four stages: hemostasis, inflammatory, proliferation, and maturation. The wound healing trajectory that can be generated can indicate the progress of a wound through the stages to healing. Further embodiments include developing and/or modifying a skin wound care plan, based on an analysis of the wound healing trajectory.

The system 1300 can include a computer program product embodied in a non-transitory computer readable medium for image analysis, the computer program product comprising code which causes one or more processors to perform operations of: obtaining one or more skin wound images for an individual, wherein the skin wound images provide light spectrum information that includes fluorescence characteristics and reflectance characteristics; analyzing, using one or more processors, the skin wound images to determine metrics for skin wound factors, based on the light spectrum information, wherein the skin wound factors comprise infection, inflammation, granulation, topology, and epithelialization; and generating an output indicative of biophysical skin wound status, wherein the output is based on the analyzing of the skin wound factors.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general-purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are limited to neither conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM); an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States, then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A method for image analysis comprising:
obtaining one or more skin wound images for an individual, wherein the skin wound images provide light spectrum information that includes fluorescence characteristics and reflectance characteristics;
analyzing, using one or more processors, the skin wound images to determine metrics for skin wound factors, based on the light spectrum information, wherein the skin wound factors comprise infection, inflammation, granulation, topology, and epithelialization; and
generating an output indicative of biophysical skin wound status, wherein the output is based on the analyzing of the skin wound factors.

2. The method of claim 1 wherein the light spectrum information includes infrared thermal characteristics.

3. The method of claim 2 further comprising augmenting the output, based on analysis of the infrared thermal characteristics.

4. The method of claim 1 wherein an epithelialization metric is included in determining skin wound topology.

5. The method of claim 4 wherein the epithelialization metric is based on detecting a nicotinamide adenine dinucleotide plus hydrogen (NADH) spatial halo around the skin wound.

6. The method of claim 1 wherein the skin wound topology comprises wound area, wound volume, wound depth, wound shape, wound edge definition, wound texture, or wound topography.

7. The method of claim 1 wherein the analyzing is based on a subset of the skin wound factors.

8. The method of claim 1 further comprising developing a skin wound care plan, based on the output.

9. The method of claim 1 wherein the metrics comprise physical and chemical skin wound signatures.

10. The method of claim 1 further comprising obtaining additional skin wound images for the individual.

11. The method of claim 10 further comprising generating a wound healing trajectory, based on the one or more skin wound images and at least one additional skin wound image.

12. The method of claim 11 further comprising developing and/or modifying a skin wound care plan, based on an analysis of the wound healing trajectory.

13. The method of claim 10 wherein the additional skin wound images provide a time-sequenced representation of skin wound healing.

14. The method of claim 1 further comprising obtaining bandage samples corresponding to the skin wound images.

15. The method of claim 14 wherein the bandage samples contain skin wound analytes.

16. The method of claim 15 further comprising analyzing the skin wound analytes to provide an analyte characterization.

17. The method of claim 16 further comprising obtaining additional bandage samples corresponding to skin wounds on a plurality of individuals.

18. The method of claim 17 further comprising analyzing the additional bandage samples to provide additional analyte characterization.

19. The method of claim 18 further comprising clustering the skin wounds on the plurality of individuals.

20. The method of claim 18 further comprising establishing analyte thresholds for inflammation, infection, granulation, and epithelialization, based on the additional analyte characterization.

21. The method of claim 16 wherein the analyte characterization is used to augment the output.

22. The method of claim 16 wherein the analyte characterization provides skin wound biochemical information.

23. The method of claim 16 wherein the analyte characterization is accomplished by analyzing a biochemical assay.

24. The method of claim 23 wherein the biochemical assay includes an immunochromatographic assay, an immunosorbent assay, or a lateral flow assay.

25. A computer program product embodied in a non-transitory computer readable medium for image analysis, the computer program product comprising code which causes one or more processors to perform operations of:
obtaining one or more skin wound images for an individual, wherein the skin wound images provide light spectrum information that includes fluorescence characteristics and reflectance characteristics;
analyzing, using one or more processors, the skin wound images to determine metrics for skin wound factors, based on the light spectrum information, wherein the skin wound factors comprise infection, inflammation, granulation, topology, and epithelialization; and
generating an output indicative of biophysical skin wound status, wherein the output is based on the analyzing of the skin wound factors.

26. A computer system for image analysis comprising:
a memory which stores instructions;
one or more processors coupled to the memory wherein the one or more processors, when executing the instructions which are stored, are configured to:
obtain one or more skin wound images for an individual, wherein the skin wound images provide light spectrum information that includes fluorescence characteristics and reflectance characteristics;
analyze, using one or more processors, the skin wound images to determine metrics for skin wound factors, based on the light spectrum information, wherein the skin wound factors comprise infection, inflammation, granulation, topology, and epithelialization; and
generate an output indicative of biophysical skin wound status, wherein the output is based on the analyzing of the skin wound factors.
